(19) United States
(12) Patent Application Publication
(10) Pub. No.: US 2023/0285512 A1
(43) Pub. Date: Sep. 14, 2023

(54) COMPOSITIONS AND METHODS FOR TREATING MYOCARDIAL INFARCTION AND ISCHEMIA

(71) Applicant: The Regents of the University of California, Oakland, CA (US)

(72) Inventors: Reza Ardehali, Los Angeles, CA (US); Ngoc Nguyen, Los Angeles, CA (US); Peng Zhao, Los Angeles, CA (US)

(21) Appl. No.: 17/693,978

(22) Filed: Mar. 14, 2022

(51) Int. Cl. A61K 38/22; A61K 9/00; A61P 9/10; C12Q 1/6883

(52) U.S. Cl. CPC A61K 38/2292; A61K 9/0019; A61P 9/10; C12Q 1/6883; C12Q 2600/158

(57) ABSTRACT

Provided herein are methods and compositions related to treating and preventing an age-related disease and inhibiting cell death using thymosin proteins.

### GOVERNMENT SUPPORT

This invention was made with government support under Grant Number HL144057, awarded by the National Institutes of Health. The government has certain rights in the invention.

### BACKGROUND

In the heart, age-related changes are important risk factors for ischemic heart disease, which is the leading cause of morbidity and mortality in the United States. Recent studies have shown that circulating factors found in young blood can partially reverse age-related loss of cognitive function, restore muscle dysfunction, and improve strength and endurance exercise capacity. In the clinical setting, it is observed that pediatric patients are able to restore baseline cardiac function after injury faster than in the aged population. Together, these studies point to the possibility that there may be specific factors in young blood that offer a protective milieu and prevent age-related degeneration. However, identification of “pro-regenerative” factors in order to design rejunevative therapies remains elusive. Thus, there remains a long-felt and unmet need for novel rejuvenative therapies for the treatment of age-related diseases including cardiovascular disease. Furthermore, new methods are needed to prevent and monitor cardiac injury.

### SUMMARY

Disclosed herein are compositions and methods related to treating or preventing an age-related disease in a subject. Such compositions and methods can be used, for example, to treat heart disease (e.g., ischemic heart disease), promote cardiac wound healing, enhance cardiac repair, reduce a humoral immune response, prevent heart failure, inhibit cardiac cell death, or prevent scarring of cardiac tissue in a subject. Accordingly, in certain embodiments, provided herein are methods of treating or preventing an age-related disease in a subject (e.g., administering a thymosin protein to the subject) and inhibiting cell death in a subject (e.g., determining whether serum of a subject comprises a level of a pro-aging factor above a threshold level and administering a thymosin protein to the subject if the level of the pro-aging factor is above the threshold level).

### BRIEF DESCRIPTION OF THE DRAWINGS

The patent application file contains at least one drawing executed in color. Copies of this patent or patent application publication with color drawing(s) will be provided by the office upon request and payment of necessary fee.

FIG. 1A shows schematic of experimental timeline in the functional and histological analysis of mouse hearts treated with neonatal plasma after I/R injury.

FIG. 1B shows ejection fraction in the functional and histological analysis of mouse hearts treated with neonatal plasma after I/R injury.

FIG. 1C shows fractional shortening at baseline and 60 days post injury in the functional and histological analysis of mouse hearts treated with neonatal plasma after I/R injury.

FIG. 1D shows trichrome staining in the functional and histological analysis of mouse hearts treated with neonatal plasma after I/R injury. Scale bars=1 mm.

FIG. 1E shows quantification to assess scar size in the functional and histological analysis of mouse hearts treated with neonatal plasma after I/R injury.

FIG. 1F shows isolectin staining in the functional and histological analysis of mouse hearts treated with neonatal plasma after I/R injury. Scale bars=1 mm.

FIG. 1G shows quantification to assess vascular density in the functional and histological analysis of mouse hearts treated with neonatal plasma after I/R injury.

FIG. 1H shows Periostin staining in the functional and histological analysis of mouse hearts treated with neonatal plasma after I/R injury. Scale bars=1 mm.

FIG. 1I shows quantification to assess for activated fibroblasts in the functional and histological analysis of mouse hearts treated with neonatal plasma after I/R injury.

FIG. 2A shows schematic of NRVM exposed to hypoxia followed by neonatal plasma treatment demonstrating the effect of neonatal plasma on the proliferation and apoptosis of cardiac cells.

FIG. 2B shows quantification of the percent of TUNEL+NRVMs demonstrating the effect of neonatal plasma on the proliferation and apoptosis of cardiac cells. * p<0.05, ** p<0.01.

FIG. 2C shows corresponding images in FIG. 2B demonstrating the effect of neonatal plasma on the proliferation and apoptosis of cardiac cells.

FIG. 2D shows percent well confluence as a measure of cellular proliferation using the Incucyte cell imaging system on HUVECs demonstrating the effect of neonatal plasma on the proliferation and apoptosis of cardiac cells.

FIG. 2E shows tubal formation assay of endothelial cell demonstrating the effect of neonatal plasma on the proliferation and apoptosis of cardiac cells.

FIG. 2F shows tubal formation assay of endothelial cell analyzed for total tube number demonstrating the effect of neonatal plasma on the proliferation and apoptosis of cardiac cells.

FIG. 2G shows tubal formation assay of endothelial cell analyzed for total branching points demonstrating the effect of neonatal plasma on the proliferation and apoptosis of cardiac cells.

FIG. 2H shows tubal formation assay of endothelial cell analyzed for total loops demonstrating the effect of neonatal plasma on the proliferation and apoptosis of cardiac cells.

FIG. 2I shows tubal formation assay of endothelial cell analyzed for total tube length demonstrating the effect of neonatal plasma on the proliferation and apoptosis of cardiac cells.

FIG. 2J shows schematic of BrdU and Annexin V flow experiment demonstrating the effect of neonatal plasma on the proliferation and apoptosis of cardiac cells.

FIG. 2K shows results of BrdU and Annexin V flow experiment of endothelial cells demonstrating the effect of neonatal plasma on the proliferation and apoptosis of cardiac cells.

FIG. 2L shows results of BrdU and Annexin V flow experiment of fibroblasts demonstrating the effect of neonatal plasma on the proliferation and apoptosis of cardiac cells.

FIG. 3A shows schematic of experimental plan of single cell RNA sequencing of mouse hearts treated with neonatal plasma.

FIG. 3B shows UMAP of cells captured from single cell RNA sequencing of mouse hearts treated with neonatal plasma.

FIG. 3C shows UMAP of identified cardiac subpopulations using known cell type markers from single cell RNA sequencing of mouse hearts treated with neonatal plasma.

FIG. 3D shows heat map of top 5 genes enriched in each experimental group from single cell RNA sequencing of mouse hearts treated with neonatal plasma.

FIG. 3E shows GO Biological Processes of the top 50 genes enriched in each experimental group of single cell RNA sequencing of mouse hearts treated with neonatal plasma.

FIG. 3F shows GO Biological Processes of the top 50 genes enriched in each experimental group with corresponding boxplots of selected pathways from single cell RNA sequencing of mouse hearts treated with neonatal plasma.

FIG. 4A shows UMAP of cells isolated from left ventricle with inset depicting cardiomyocyte subpopulation from single cell RNA sequencing analysis of cardiomyocyte subpopulations.

FIG. 4B shows UMAP of cardiomyocyte subpopulation labeled by treatment group from single cell RNA sequencing analysis of cardiomyocyte subpopulations.

FIG. 4C shows FeaturePlot of Tnnt2 expression from single cell RNA sequencing analysis of cardiomyocyte subpopulations.

FIG. 4D shows Dotplot (left) of top 10 genes and corresponding GO Biological Process of the top 50 genes enriched in each treatment group from single cell RNA sequencing analysis of cardiomyocyte subpopulations.

FIG. 4E shows UMAP of cardiomyocyte subpopulation labeled by clusters from single cell RNA sequencing analysis of cardiomyocyte subpopulations.

FIG. 4F shows quantification of the proportion of cells from each treatment group within each cluster from single cell RNA sequencing analysis of cardiomyocyte subpopulations.

FIG. 4G shows Heatmap of the top 8 genes from each cardiomyocyte cluster from single cell RNA sequencing analysis of cardiomyocyte subpopulations.

FIG. 4H shows Heatmap of the top 8 genes from each cardiomyocyte cluster identified with the top 4 genes displayed as FeaturePlot from single cell RNA sequencing analysis of cardiomyocyte subpopulations.

FIG. 4I shows Heatmap of the top 8 genes from each cardiomyocyte cluster identified with the GO Biological process of the top 100 genes from each cluster from single cell RNA sequencing analysis of cardiomyocyte subpopulations.

FIG. 5A shows plasma of neonatal mice 2-5 days and adult mice 1 year of age were obtained for mass spectrometry.

FIG. 5B shows 872 proteins were identified, of which 310 were increased (>2-fold) and 85 were decreased (<0.5-fold) in abundance in neonatal compared to adult plasma from mass spectrometry of neonatal and aged plasma.

FIG. 5C shows list and corresponding heatmaps of the top 15 decreased abundance proteins found in neonatal plasma compared to adult plasma from mass spectrometry of neonatal and aged plasma.

FIG. 5D shows list and corresponding heatmaps of the top 15 increased abundance proteins found in neonatal plasma compared to adult plasma from mass spectrometry of neonatal and aged plasma. Thymosin proteins Tmsb4x, Tmsb10, and Ptma are highlighted.

FIG. 5E shows volcano plot of proteins identified from mass spectrometry of neonatal and aged plasma.

FIG. 5F shows GO Biological Process of decreased abundance proteins from mass spectrometry of neonatal and aged plasma.

FIG. 5G shows GO Biological Process of increased abundance proteins from mass spectrometry of neonatal and aged plasma.

FIG. 5H shows analysis of thymosin proteins Tmsb4x, Tmsb10, and Ptma.

FIG. 6A shows live/dead analysis of HL-1 cardiomyocytes with thymosin (34 from in vitro assessment of hypoxia-induced apoptosis and treatment with protein candidates in HL-1 cardiomyocytes.

FIG. 6B shows live/dead analysis of HL-1 cardiomyocytes with thymosin (310 from in vitro assessment of hypoxia-induced apoptosis and treatment with protein candidates in HL-1 cardiomyocytes.

FIG. 6C shows live/dead analysis of HL-1 cardiomyocytes with prothymosin α under normoxic and hypoxic conditions from in vitro assessment of hypoxia-induced apoptosis and treatment with protein candidates in HL-1 cardiomyocytes.

FIG. 6D shows effect on cell viability of HL-1 cardiomyocytes to varying dose of thymosin (34 from in vitro assessment of hypoxia-induced apoptosis and treatment with protein candidates in HL-1 cardiomyocytes.

FIG. 6E shows effect on cell viability of HL-1 cardiomyocytes to varying dose of thymosin (310 from in vitro assessment of hypoxia-induced apoptosis and treatment with protein candidates in HL-1 cardiomyocytes.

FIG. 6F shows effect on cell viability of HL-1 cardiomyocytes to varying dose of prothymosin α from in vitro assessment of hypoxia-induced apoptosis and treatment with protein candidates in HL-1 cardiomyocytes.

FIG. 6G shows quantification of the number of DAPI+ cells per field after treatment with thymosin β4.

FIG. 6H shows quantification of the number of DAPI+ cells per field after treatment with thymosin β10.

FIG. 6I shows quantification of the number of DAPI+ cells per field after treatment with prothymosin α.

FIG. 7 shows tubal formation assay of endothelial cell demonstrating the effect of neonatal plasma on the proliferation and apoptosis of cardiac cells.

FIG. 8A shows representative M-mode echocardiographic images of hearts 60 days following injury and treatment.

FIG. 8B shows scar size as assessed by midline length.

FIG. 8C shows scar size as assessed by infarct wall thickness

FIG. 8D shows heart weights normalized to body weight.

FIG. 8E shows heart weights normalized to tibia length.

FIG. 9A shows Incucyte images of endothelial cells treated with reduced serum media (1% FBS, 3% FBS), denatured neonatal plasma, or neonatal plasma at (top) baseline, (middle) 12 hours, and (bottom) 28 hours demonstrating effect of neonatal plasma on the proliferation and apoptosis of endothelial and fibroblasts.

FIG. 9B shows Analysis of percent BrdU+ and Annexin V+ within fibroblasts and endothelial cells treated with neonatal plasma or saline demonstrating effect of neonatal plasma on the proliferation and apoptosis of endothelial and fibroblasts.

FIG. 10A shows UMAP of cells isolated from whole heart, split into separate treatment groups from identification and verification of cardiac subpopulations using established cell type markers.

FIG. 10B shows FeaturePlot of known cell type markers for identification of cardiac subpopulations.

FIG. 10C shows Verification of selected cardiac subpopulations using dotplot (left) of top genes within each cluster and corresponding GO Biological Process of the top 100 genes within each subpopulation (right) from identification and verification of cardiac subpopulations using established cell type markers.

FIG. 11A shows schematic of mass spectrometry process.

FIG. 11B shows protein mass from categorization of proteins identified from mass spectrometry.

FIG. 11C peptide length distribution of total, increased, and decreased abundance proteins from categorization of proteins identified from mass spectrometry.

FIG. 11D shows component analysis of plasma samples to show variability across biological samples from categorization of proteins identified from mass spectrometry.

FIG. 11E shows component analysis of plasma samples to show variability across biological samples from categorization of proteins identified from mass spectrometry.

FIG. 12A shows GO term classification of thymosin protein candidates from database search and comparison of mass spectrometry results.

FIG. 12B shows comparison of mass spectrometry results with Yang et al study from database search and comparison of mass spectrometry results.

FIG. 12C shows gene expression trends across different organs and developmental age of candidates using the Kaessmann database from database search and comparison of mass spectrometry results.

FIG. 13 shows a schematic representation of in vivo study.

### DETAILED DESCRIPTION

### General

The present disclosure relates to methods and compositions for treating or preventing an age-related disease and/or inhibiting cell death in a subject (e.g., administering a thymosin protein to the subject). Such methods may optionally comprise determining whether serum of a subject comprises a level of a pro-aging factor above a threshold level and administering a thymosin protein to the subject if the level of the pro-aging factor is above the threshold level. The methods and compositions provided herein are based, in part, on the discovery that cardiac cells can be effectively treated with a thymosin protein (e.g., recombinant thymosin protein), thereby inhibiting cardiac cell death that eventually leads to cardiac injury and heart disease. Exemplary thymosin proteins include Tmsb4x (Thymosin beta 4), Tmsb10 (Thymosin beta 10), and Ptma (Prothymosin alpha). In certain aspects, the methods and compositions provided herein may be advantageously used to treat cardiac injury conjointly with another therapeutic agent. For example, in certain embodiments the methods and compositions provided herein may be used to treat cardiac injury conjointly with an additional thymosin protein (e.g., Tmsb4x, Tmsb10, or Ptma) and/or an additional cardiovascular therapeutic.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” refers to any substance, compound (e.g., molecule), supramolecular complex, material, or combination or mixture thereof.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

The term “binding” or “interacting” refers to an association, which may be a stable association, between two molecules, due to, for example, electrostatic, hydrophobic, ionic and/or hydrogen-bond interactions under physiological conditions.

In certain embodiments, therapeutic compounds may be used alone or conjointly administered with another type of therapeutic agent (e.g., an additional thymosin protein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional cardiovascular therapeutic agent(s)) provides improved efficacy relative to each individual administration of the compound (e.g., thymosin protein) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

The term “measuring” refers to determining the presence, absence, quantity amount, or effective amount of a substance in a sample, including the concentration levels of such substances.

As used herein, the term “subject” means a human or non-human animal selected for treatment or therapy.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “cardiomyopathy” refers to any disease or dysfunction of the myocardium (heart muscle) in which the heart is abnormally enlarged, thickened and/or stiffened. As a result, the heart muscle's ability to pump blood is usually weakened. The etiology of the disease or disorder may be, for example, inflammatory, metabolic, toxic, infiltrative, fibroplastic, hematological, genetic, or unknown in origin. There are two general types of cardiomyopathies: ischemic (resulting from a lack of oxygen) and non-ischemic.

As used herein, “chronic heart failure” or “congestive heart failure” or “CHF” refer, interchangeably, to an ongoing or persistent forms of heart failure. Common risk factors for CHF include old age, diabetes, high blood pressure and being overweight. CHF is broadly classified according to the systolic function of the left ventricle as HF with reduced or preserved ejection fraction (HFrEF and HFpEF). The term “heart failure” does not mean that the heart has stopped or is failing completely, but that it is weaker than is normal in a healthy person. In some cases, the condition can be mild, causing symptoms that may only be noticeable when exercising. In others, the condition may be more severe, causing symptoms that may be life-threatening, even while at rest. The most common symptoms of chronic heart failure include shortness of breath, tiredness, swelling of the legs and ankles, chest pain and a cough. In some embodiments, the methods of the disclosure decrease, prevent, or ameliorate one or more symptoms of CHF (e.g., HFrEF) in a subject suffering from or at risk for CHF (e.g., HFrEF). In some embodiments, the disclosure provides methods of treating CHF and conditions that can lead to CHF.

As used herein “acute heart failure” (AHF) or “decompensated heart failure” refer, interchangeably, to a syndrome of the worsening of signs and symptoms reflecting an inability of the heart to pump blood at a rate commensurate to the needs of the body at normal filling pressure. AHF typically develops gradually over the course of days to weeks and then decompensates requiring urgent or emergent therapy due to the severity of these signs or symptoms. AHF may be the result of a primary disturbance in the systolic or diastolic function of the heart or of abnormal venous or arterial vasoconstriction, but generally represents an interaction of multiple factors, including volume overload. The majority of patients with AHF have decompensation of chronic heart failure (CHF) and consequently much of the discussion of the pathophysiology, presentation, and diagnosis of CHF is directly relevant to an understanding of AHF. In other cases, AHF results from an insult to the heart or an event that impairs heart function, such as an acute myocardial infarction, severe hypertension, damage to a heart valve, abnormal heart rhythms, inflammation or infection of the heart, toxins and medications. In some embodiments, the methods of the disclosure decrease, prevent, or ameliorate one or more symptoms of AHF in a subject suffering from or at risk for AHF. In some embodiments, the disclosure provides methods of treating AHF and conditions that can lead to AHF. AHF may be the result of ischemia associated with myocardial infarction.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac alpha-actinin. The term “cardiomyocyte” as used herein is an umbrella term comprising any cardiomyocyte subpopulation or cardiomyocyte subtype, e.g., atrial, ventricular and pacemaker cardiomyocytes.

As used herein, the term “thymosin protein” refers to a group of small peptides with molecular weights of 1000-15,000 Da that were originally isolated from the thymus gland. Thymosin proteins are present in a variety of mammalian tissues and are biological response modifiers. Thymosin proteins are involved in modulating and regulating cell migration, angiogenesis, immune responses, and tissue regeneration. Exemplary thymosin proteins include Tmsb4x (Thymosin beta 4), Tmsb10 (Thymosin beta 10), and Ptma (Prothymosin alpha).

### Pharmaceutical Compositions and Administration

In certain embodiments, provided herein are pharmaceutical compositions and methods of using pharmaceutical compositions. In some embodiments, the pharmaceutical compositions provided herein comprise a thymosin protein (e.g., Tmsb4x, Tmsb10, or Ptma). In some embodiments, the pharmaceutical compositions provided herein comprise an additional therapeutic agent (e.g., an additional thymosin protein or additional cardiovascular therapeutic).

In certain embodiments, the compositions and methods provided herein may be utilized to treat a subject in need thereof. The subject may be a mammal such as a human, or a non-human mammal. In some embodiments, the subject has an age-related disease (e.g., heart disease). In certain embodiments, the compositions and methods provided herein may be utilized to promote cardiac wound healing, enhance cardiac repair, reduce a humoral immune response, prevent heart failure, inhibit cardiac cell death, or prevent scarring of cardiac tissue

When administered to a subject, such as a human, the composition or the compound is preferably administered as a pharmaceutical composition comprising, for example, a therapeutic compound and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, the pharmaceutical compositions provided herein comprise a pharmaceutically acceptable carrier. The phrase “pharmaceutically acceptable carrier” as used herein means a pharmaceutically acceptable material, composition or vehicle, such as a liquid or solid filler, diluent, excipient, solvent or encapsulating material. A pharmaceutically acceptable carrier can contain physiologically acceptable agents that act, for example, to stabilize, increase solubility or to increase the absorption of a compound. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, the pharmaceutical compositions provided herein can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In certain embodiments, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular subject, composition, and mode of administration, without being toxic to the subject.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In some embodiments, the active compound will be administered once daily.

In certain embodiments, compounds may be used alone or conjointly administered with another type of therapeutic agent (e.g., an additional thymosin protein or an additional cardiovascular therapeutic disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional chemotherapeutic agent(s)) provides improved efficacy relative to each individual administration of the compound (e.g., thymosin protein) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

Pharmaceutically acceptable salts of compounds in the methods provided herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn, copper, cobalt, cadmium, manganese, or other metal salts.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the therapeutic compound used in the methods herein is a thymosin protein. Exemplary thymosin proteins are listed in Table 1. In some embodiments, the thymosin protein is a recombinant thymosin protein.

TABLE 1
Exemplary Thymosin Proteins

| | NCBI | |
|---|---|---|
| | Reference | |
| | Sequence | NCBI Amino Acid |
| Compound Name | (Human) | Sequence (Human) |
| Tmsb4x (Thymosin | NP_066932 | MSDKPDMAEIEKFDKSK |
| beta 4) | | LKKTETQEKNPLPSKETI |
| | | EQEKQAGES |
| Tmsb10 (Thymosin | NP_066926 | MADKPDMGEIASFDKAK |
| beta 10) | | LKKTETQEKNTLPTKETI |
| | | EQEKRSEIS |
| Ptma (Prothymosin | NP_001092755 | MSDAAVDTSSEITTKDL |
| alpha) | | KEKKEVVEEAENGRDAP |
| isoform 1 | | ANGNAENEENGEQEAD |
| | | NEVDEEEEEGGEEEEEEE |
| | | EGDGEEEDGDEDEEAES |
| | | ATGKRAAEDDEDDDVD |
| | | TKKQKTDEDD |
| Ptma (Prothymosin | NP_002814 | MSDAAVDTSSEITTKDL |
| alpha) | | KEKKEVVEEAENGRDAP |
| isoform 2 | | ANGNANEENGEQEADN |
| | | EVDEEEEEGGEEEEEEEE |
| | | GDGEEEDGDEDEEAESA |
| | | TGKRAAEDDEDDDVDT |
| | | KKQKTDEDD |

In some embodiments, the thymosin protein may be administered conjointly with an additional thymosin protein (e.g., a recombinant thymosin protein). For example, prothymosin α and thymosin β4 may be conjointly administered to a subject.

In some embodiments, the thymosin protein may be administered conjointly with an additional therapeutic compound such as an additional cardiovascular therapeutic agent. Exemplary classes of additional cardiovascular therapeutic agents include beta blockers, ACE inhibitors, angiotensin receptor blockers, aldosterone antagonist, digoxin, hydralazine and nitrates, and diuretics. Examples of additional cardiovascular therapeutic agents include, but are not limited to, sulfaphenazole, chloramphenicol, statins, metformin, resveratrol, minoxidil, clonidine, amiodarone, intermedin, enalapril, candesartan, spironolactone, pravastin, atorvastin, dexrazoxane, aspirin, enoxaparin, rivaroxaban/apixaban, carvedilol, nebivolol, metoprolol, bisoprilol, lisinopril, captopril, losartan, entresto, sacubitril/valsartan, spironolactone, eplerenone, Apresoline, Nitrobid, Imdur, Isordil, furosemide (Lasix), bumetanide (Bumex), torsemide (Demadex), and metolazone (Zaroxolyn).

Actual dosage levels of the therapeutic compound may be varied so as to obtain an amount which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

### Methods of Treating or Preventing Age-Related Disease

In certain aspects, provided herein are methods of treating or preventing an age-related disease in a subject by administering to the subject a therapeutic compound according to a method provided herein. In certain embodiments, the therapeutic compound is a thymosin protein.

### Immune Disorders

Exemplary age-related diseases include diseases associated with a pathological immune response. The compositions described herein can be used, for example, for preventing or treating an autoimmune disease, such as chronic inflammatory bowel disease, systemic lupus erythematosus, psoriasis, rheumatoid arthritis, multiple sclerosis, or Hashimoto's disease; or an infectious disease, such as an infection with Streptococcus pneumonia (e.g., age-related Streptococcus pneumonia infection).

In some embodiments, the compositions and methods provided herein are useful for the treatment or prevention of age-related inflammation. In certain embodiments, the pharmaceutical compositions described herein can be used for preventing or treating inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the compositions and methods described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to an immune disorder that affects any structure of the eye, including the eye lids. Examples of ocular immune disorders which may be treated with the compositions and methods described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the compositions and methods described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the compositions and methods described herein include, but are not limited to, arthrosclerosis, arthritis, phlebitis, vasculitis, and lymphangitis.

Examples of digestive system immune disorders which may be treated with the compositions and methods described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the compositions and methods described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The compositions and methods described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

### Metabolic Disorders

In some embodiments, the compositions and methods described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the compositions and methods described herein relate to the treatment of Nonalcoholic Fatty Liver Disease (NAFLD) and Nonalcoholic Steatohepatitis (NASH).

The compositions and methods described herein can be used to treat any subject in need thereof.

### Liver Disease

In some embodiments, the compositions and methods described herein relate to the treatment of liver diseases. Such diseases include, but are not limited to, Alcohol-Related Liver Disease, Autoimmune Hepatitis, Cirrhosis, Hepatitis A, Hepatitis B, Hepatitis C, Hepatic Encephalopathy, Intrahepatic Cholestasis of Pregnancy (ICP), and Liver Cysts.

### Neurodegenerative Disease

The compositions and methods and/or solid dosage forms described herein may be used to treat neurodegenerative and neurological diseases. In certain embodiments, the neurodegenerative and/or neurological disease is Parkinson's disease, Alzheimer's disease, prion disease, Huntington's disease, macular degeneration, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Cardiovascular Disease

In some embodiments, the compositions and methods described herein relate to the treatment or prevention of heart diseases, vascular diseases and/or cardiovascular diseases or disease of the cardiovascular system. For example, the compositions described herein relate to the treatment or prevention of acute and chronic heart failure, arterial hypertension, coronary heart disease, stable and instable angina pectoris, myocardial ischemia, myocardial infarction, coronary microvascular dysfunction, microvascular obstruction, no-reflow-phenomenon, shock, atherosclerosis, coronary artery disease, peripheral artery disease, peripheral arterial disease, intermittent claudication, severe intermittent claudication, limb ischemia, critical limb ischemia, hypertrophy of the heart, cardiomyopathies of any etiology (such as, e.g., dilatative cardiomyopathy, restrictive cardiomyopathy, hypertrophic cardiomyopathy, ischemic cardiomyopathy), fibrosis of the heart, atrial and ventricular arrhythmias, transitory and/or ischemic attacks, apoplexy, ischemic and/or hemorrhagic stroke, preeclampsia, inflammatory cardiovascular diseases, metabolic diseases, diabetes, type-I-diabetes, type-II-diabetes, diabetes mellitus, peripheral and autonomic neuropathies, diabetic neuropathies, diabetic microangiopathies, diabetic retinopathy, diabetic ulcera at the extremities, gangrene, CREST-syndrome, hypercholesterolemia, hypertriglyceridemia, lipometabolic disorder, metabolic syndrome, increased levels of fibrinogen and low-density lipoproteins (i.e. LDL), increased concentrations of plasminogen-activator inhibitor 1 (PAI-1), as well as peripheral vascular and cardiac vascular diseases, peripheral circulatory disorders, primary and secondary Raynaud syndrome, disturbances of the microcirculation, arterial pulmonary hypertension, spasms of coronary and peripheral arteries, thromboses, thromboembolic diseases, edema-formation, such as pulmonary edema, brain-edema, renal edema, myocardial edema, myocardial edema associated with heart failure, restenosis after i.e. thrombolytic therapies, percutaneous-transluminal angioplasties (PTA), transluminal coronary angioplasties (PTCA), heart transplantations, bypass-surgeries as well as micro- and macrovascular injuries (e.g., vasculitis), reperfusion-damage, arterial and venous thromboses, microalbuminuria, cardiac insufficiency, endothelial dysfunction. In the light of the present disclosure, heart failure includes more specific or related kinds of diseases such as acute decompensated heart failure, right heart failure, left heart failure, global insufficiency, ischemic cardiomyopathy, dilatative cardiomyopathy, congenital heart defect(s), valve diseases, heart failure related to valve diseases, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined valvular defects, inflammation of the heart muscle (myocarditis), chronic myocarditis, acute myocarditis, viral myocarditis, bacterial myocarditis, diabetic heart failure, alcohol-toxic cardiomyopathy, cardiac storage diseases, heart failure with preserved ejection fraction (HFpEF), diastolic heart failure, heart failure with reduced ejection fraction (HFrEF), systolic heart failure, Atrial fibrillation, paroxysmal atrial fibrillation, intermittent atrial fibrillation, persistent atrial fibrillation, permanent atrial fibrillation, atrial flutter, sinus arrhythmia, sinus tachycardia, passive heterotopy, active heterotopy, replacement systoles, extrasystoles, disturbances in the conduction of impulses, sick-sinus syndrome, hypersensitive carotis-sinus, tachycardias, AV-node re-entry tachycardias, atrioventricular re-entry tachycardia, WPW-syndrome (Wolff-Parkinson-White syndrome), Mahaim-tachycardia, hidden accessory pathways/tracts, permanent junctional re-entry tachycardia, focal atrial tachycardia, junctional ectopic tachycardia, atrial re-entry tachycardia, ventricular tachycardia, ventricular flutter, ventricular fibrillation, sudden cardiac death. In the context of the present disclosure, the term coronary heart disease also include more specific or related diseases entities, such as: Ischemic heart disease, stable angina pectoris, acute coronary syndrome, instable angina pectoris, NSTEMI (non-ST-segement-elevation myocardial infarction), STEMI (ST-segement-elevation myocardial infarction), ischemic damage of the heart, arrhythmias, and myocardial infarction.

In certain embodiments, the compositions and methods provided herein may be utilized to improve cardiac function and/or increase vascular density in the heart after ischemia-reperfusion injury.

In certain embodiments, the compositions and methods provided herein may be utilized to reduce scar size in the heart of a subject without pre-existing scar tissue compared to non-treatment of the subject. For example, the compositions and methods provided herein may be used to reduce scar size in the heart of a subject without pre-existing scar tissue following ischemia-reperfusion injury.

Methods of Inhibiting Cell death

In certain aspects, provided herein are methods related to inhibiting cell death (e.g., cardiac cell death) in a subject, comprising: (a) determining whether serum of a subject comprises a level of a pro-aging factor above a threshold level; and (b) if the level of the pro-aging factor is above the threshold level, administering a thymosin protein to the subject.

In some embodiments, pro-aging factor is encoded by a gene selected from the group consisting of Crct1, Sprr1a, Serpinb1a, and Lgals3.

In certain embodiments, determining whether serum of a subject comprises a level of a pro-aging factor above a threshold level comprises measuring the level of the pro-aging factor in serum of the subject.

In certain embodiments, the threshold level of the pro-aging factor in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise pro-aging factor.

In some embodiments, any assay capable of detecting levels of the relevant pro-aging factor (a biomarker) can be used in the methods provided herein. In some embodiments, the pro-aging factor is detected by immunostaining with a labeled antibody that binds to the biomarker epitope. In some embodiments, the biomarker is detected by immunohistochemistry. In some embodiments, the biomarker is detected by Western Blot. In some embodiments, the mRNAs of the biomarker are detected using qPCR. In some embodiments, the biomarker is detected using fluorescence activated cell sorting (FACS). In some embodiments, the biomarker is detected using microscopy (e.g., fluorescence microscopy). In some embodiments, the biomarker is detected using ELISA.

Any of a variety of antibodies can be used in methods of the detection. Such antibodies include, for example, polyclonal, monoclonal (mAbs), recombinant, humanized or partially humanized, single chain, Fab, and fragments thereof. The antibodies can be of any isotype, e.g., IgM, various IgG isotypes such as IgG1, IgG2a, etc., and they can be from any animal species that produces antibodies, including goat, rabbit, mouse, chicken or the like. The term “an antibody specific for” a protein means that the antibody recognizes a defined sequence of amino acids, or epitope, in the protein, and binds selectively to the protein and not generally to proteins unintended for binding to the antibody. The parameters required to achieve specific binding can be determined routinely, using conventional methods in the art.

In some embodiments, antibodies specific for a biomarker (e.g., pro-aging factor) are immobilized on a surface (e.g., are reactive elements on an array, such as a microarray, or are on another surface, such as used for surface plasmon resonance (SPR)-based technology, such as Biacore), and proteins in a sample are detected by virtue of their ability to bind specifically to the antibodies. Alternatively, proteins in the sample can be immobilized on a surface, and detected by virtue of their ability to bind specifically to the antibodies. Methods of preparing the surfaces and performing the analyses, including conditions effective for specific binding, are conventional and well-known in the art.

Among the many types of suitable immunoassays are immunohistochemical staining, ELISA, Western blot (immunoblot), immunoprecipitation, radioimmunoassay (RIA), fluorescence-activated cell sorting (FACS), etc. In some embodiments, assays used in methods provided herein can be based on colorimetric readouts, fluorescent readouts, mass spectroscopy, visual inspection, etc.

As mentioned above, expression levels of a biomarker can be measured by measuring nucleic acid amounts (e.g., mRNA amounts and/or genomic DNA). The determination of nucleic acid amounts can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Copy number changes may be detected, for example, with the Illumina Infinium II whole genome genotyping assay or Agilent Human Genome CGH Microarray (Steemers et al., 2006). Examples of methods to measure mRNA amounts include reverse transcriptase-polymerase chain reaction (RT-PCR), including real time PCR, microarray analysis, nanostring, Northern blot analysis, differential hybridization, and ribonuclease protection assay. Such methods are well-known in the art and are described in, for example, Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & sons, New York, N.Y.

### Methods of Screening Candidate Pro-regenerative Factors

Certain aspects of the disclosure are directed to a method of screening one or more test agents to identify a candidate pro-regenerative factor, comprising contacting a cell sample (e.g., cardiac cell) with a test agent, measuring a level of hypoxia of the cell sample and identifying the test agent as a candidate pro-regenerative factor if the level of hypoxia is decreased as compared to a level of hypoxia of a corresponding cell sample not contacted with the test agent. The level of hypoxia of a corresponding cell sample not contacted with the test agent can be any suitable reference, such as a control sample or a reference sample.

In some embodiments, the method further comprises measuring cell death of the contacted cell sample and determining if cell death of the contacted cell is decreased as compared to cell death of a corresponding cell sample not contacted with the test agent.

In some embodiments, any assay capable of detecting cell death after treatment with a test agent can be used in the methods provided herein. Cell death is typically characterized by membrane blebbing, condensation of cytoplasm, and the activation of endogenous endonucleases.

In some embodiments, any assay capable of detecting cell death after treatment with a test agent can be used in the methods provided herein. Cell death is typically characterized by membrane blebbing, condensation of cytoplasm, and the activation of endogenous endonucleases.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

Cell death can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Cell death can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring cell death number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine cell death include, e.g., cytoplasmic condensation, increased membrane blebbing, and cellular shrinkage.

The presence of cell death can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting cell death (e.g., by measuring DNA fragmentation). (See, e.g., Piazza et al., 1995, Cancer Research 55:3110-16).

### EXEMPLIFICATION

In the heart, age-related changes are important risk factors for ischemic heart disease, which is the leading cause of morbidity and mortality in the United States. Recent studies have shown that circulating factors found in young blood can partially reverse age-related loss of cognitive function, restore muscle dysfunction, and improve strength and endurance exercise capacity. In the clinical setting, it is observed that pediatric patients are able to restore baseline cardiac function after injury faster than in the aged population. Given these observations, as well as the known ability of the heart to regenerate after apical resection within the first 7 days of life, the study examined whether neonatal plasma contains “pro-youthful” factors that offer a protective milieu and prevent irreversible myocardial damage in adult mice after ischemia-reperfusion injury. The study observed reduced scar sizes, improved cardiac function, and increased vascular density in hearts of adult C57BL/6 mice treated with neonatal plasma two months post-injury. Neonatal plasma also reduced the percentage of TUNEL+ cardiomyocytes after exposure to hypoxia and improved the angiogenic ability of endothelial cells as measured by tubal formation assay. Single cell RNA sequencing and gene ontology analysis revealed a possible role of neonatal plasma in downregulation of apoptosis-related pathways. Mass spectrometry identified several thymosin-related proteins to be differentially abundant between neonatal and old plasma. Among many factors, several thymosin-related proteins were deemed promising. Validation studies using an in vitro hypoxia assay showed reduced numbers of apoptotic cardiomyocytes when treated with recombinant forms of these candidate proteins. The study offers critical insights into a more relevant neonatal period for assessment of young circulating factors and highlights potential beneficial role(s) of thymosin proteins in ischemic heart disease. This study also brings to light the potential of neonatal plasma as a rejuvenative therapy for cardiovascular as well as other age-related diseases.

### Introduction

It is well established that aging drives the impairment and degenerative processes of various organ systems within the body. Much of this has been attributed to reduced responsiveness of stem/progenitor cells, particularly within muscle, blood, liver, and brain. These critical cell types, with their ability to self-renew and produce new adult cells, play a vital role in the maintenance of normal tissue function and regeneration in response to injury or disease. For instance, studies suggest that the decline in skeletal muscle function and mass with age is due to reduced ability of muscle satellite/progenitor cells to regenerate muscle fibers. Other cell types previously associated with low regeneration rates are cardiomyocytes in the heart and neurons of the central nervous system, both of which are classified as terminally differentiated and precluded from re-entering the cell cycle. There is a general consensus that the existence of endogenous cardiac stem cells is very limited and unlikely to be a source of cardiac regeneration. Recent studies have shown that adult cardiomyocytes can be stimulated to proliferate under specified cues, albeit at low rates. These pivotal studies have now shifted the focus toward finding ways to induce the proliferation of existing cardiomyocytes as a means of cardiac therapy.

On the flip side, young age is generally associated with high regenerative ability and increased cellular plasticity. In the context of the heart, it has been shown that fetal and neonatal cardiomyocytes have the ability to proliferate and that cardiac regeneration in the face of injury occurs without scar formation. While the cellular mechanisms driving these changes are not well understood, researchers have pondered whether exposure of aged cells to a young environment can reverse the degenerative processes associated with aging. In recent years, there has been a renewed interest in examining this phenomenon, in which researchers have tuned to parabiosis as a model to examine effects of shared circulation. Much of the initial studies were within the neurology field in which heterochronic parabiosis showed that exposure to young circulation improves long-term potentiation of the dentate gyrus, enhanced learning, memory, and cognitive function, remyelination, as well as vascular remodeling and neurogenesis within the aged mouse. It is postulated that the beneficial effects of young plasma were likely soluble, heat-labile factors as heat denaturation mitigated these effects. These studies fueled further investigations within other organ systems, particularly in the muscle and cardiac fields. Conboy et al examined the efficacy of muscle regeneration in young and aged mice in heterochronic and isochronic pairings after hind limb injury. They noted that parabiosis with young mice significantly enhanced the regeneration of muscle in old partners and induced muscle stem cell activation, with the appearance of nascent myotubes similar to those found in young mice. In addition to improving regeneration in response to injury, exposure to young systemic environment was shown to reverse age-related pathology. Loffredo et al examined hearts of heterochronic pairings and noted a reversal of age-related cardiac hypertrophy after 4 weeks.

Together, these studies point to the possibility that there may be specific factors in young blood that offer a protective milieu and prevent age-related degeneration. However, the identification of these “pro-regenerative” factors remains elusive. Of note, prior studies have categorized “young circulation” to a much later period of development during which “pro-regenerative” factors may have already declined. In this study, the study aim to understand the role of systemic factors in the context of cardiac ischemia-reperfusion injury, with a focus on the neonatal period during which the heart's ability to fully regenerate after injury is well established¹⁷. The results show that neonatal plasma significantly improved cardiac function and scar sizes of adult mice after ischemia-reperfusion injury and that these effects may be mediated by thymosin proteins via apoptosis-associated pathways. The study highlights a distinct proteomic profile of neonatal plasma and brings to light its potential as a rejuvenative therapy for cardiovascular as well as other age-related diseases.

### Results

Neonatal Plasma Improves Cardiac Function, Reduces Scar Size, and Increases Vascular Density of Mouse Hearts after I/R Injury

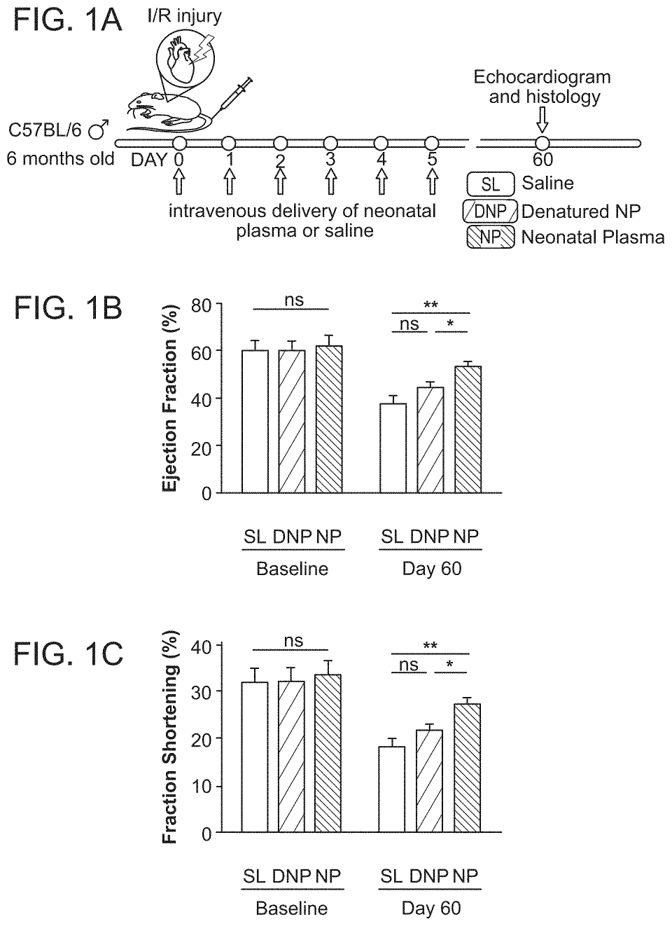

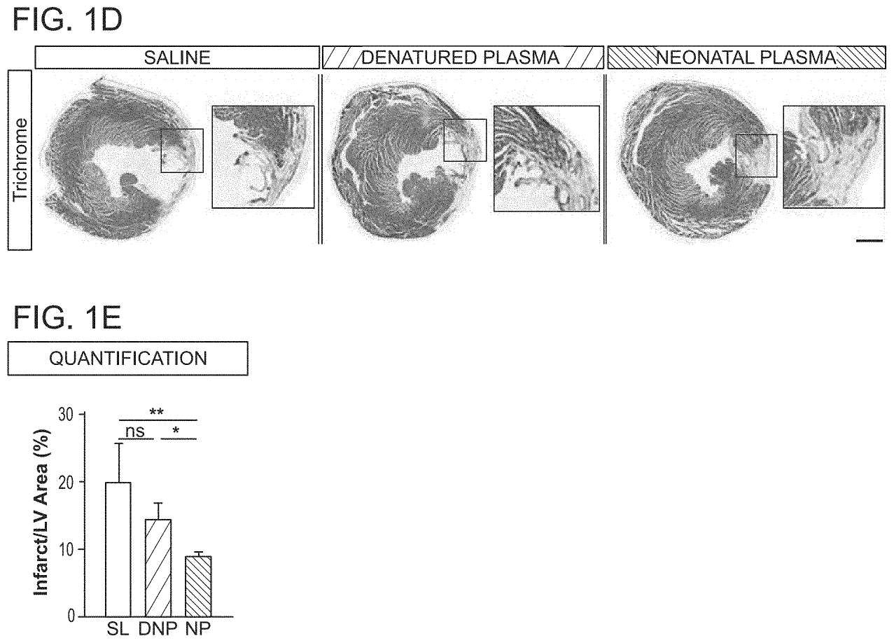

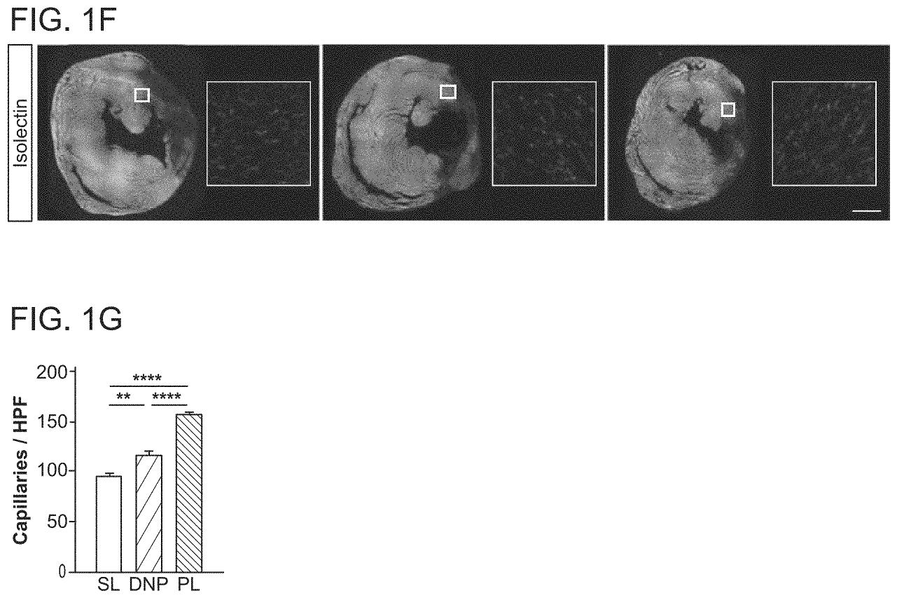

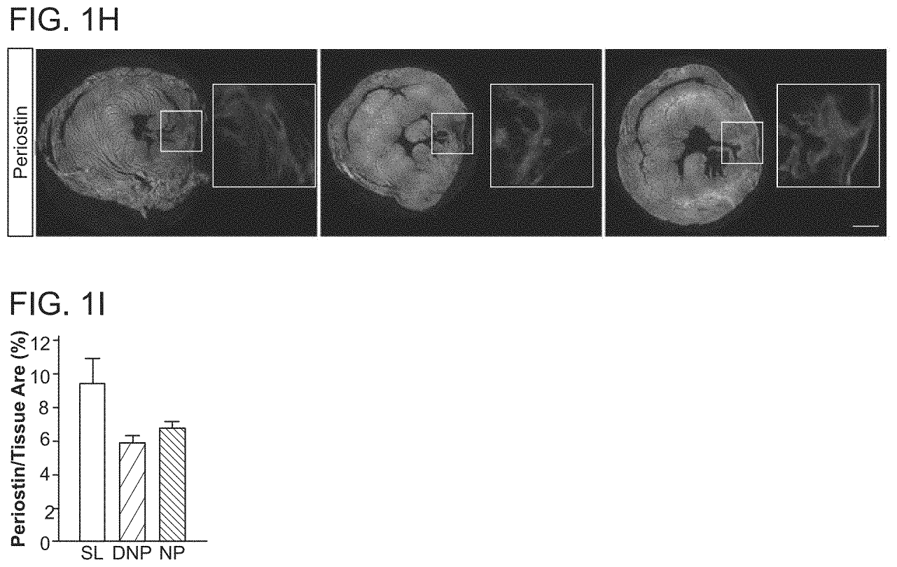

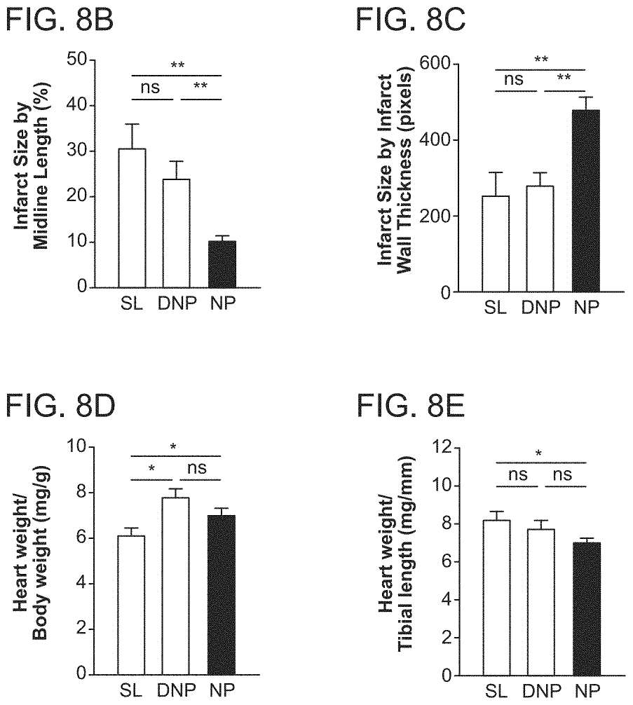

To determine whether systemic factors found in neonatal plasma may offer protection from scar formation and heart failure after ischemic injury, experimental I/R on mice 6 months of age was induced by ligation of the left anterior descending artery (LAD) and release after 45 minutes. Neonatal plasma collected from mice 2-5 days old were intravenously administered on day of surgery and for 5 consecutive days following (FIG. 1a). Functional studies at baseline and 60 days post injury showed improvement in ejection fraction and fractional shortening in groups receiving neonatal plasma, whereas denatured neonatal plasma and saline controls offered no significant protection from left ventricular dysfunction (FIG. 1b,c and FIG. 8a). Additionally, assessment of fibrosis by Masson's trichrome revealed a reduction in infarct size in mice receiving neonatal plasma (FIG. 1d,e and FIG. 8b,c). Quantification of isolectin at border zones as assessment of capillary density showed a significant increase in the number of capillaries in hearts treated with neonatal plasma compared to saline or denatured plasma controls (FIG. 1f,g). Despite the decrease in scar sizes, neonatal plasma treatment did not affect periostin expression, a marker of activated fibroblasts (FIG. 1h,i).

### Neonatal Plasma Ameliorates Apoptosis and Enhances Proliferation of Cardiac Cells

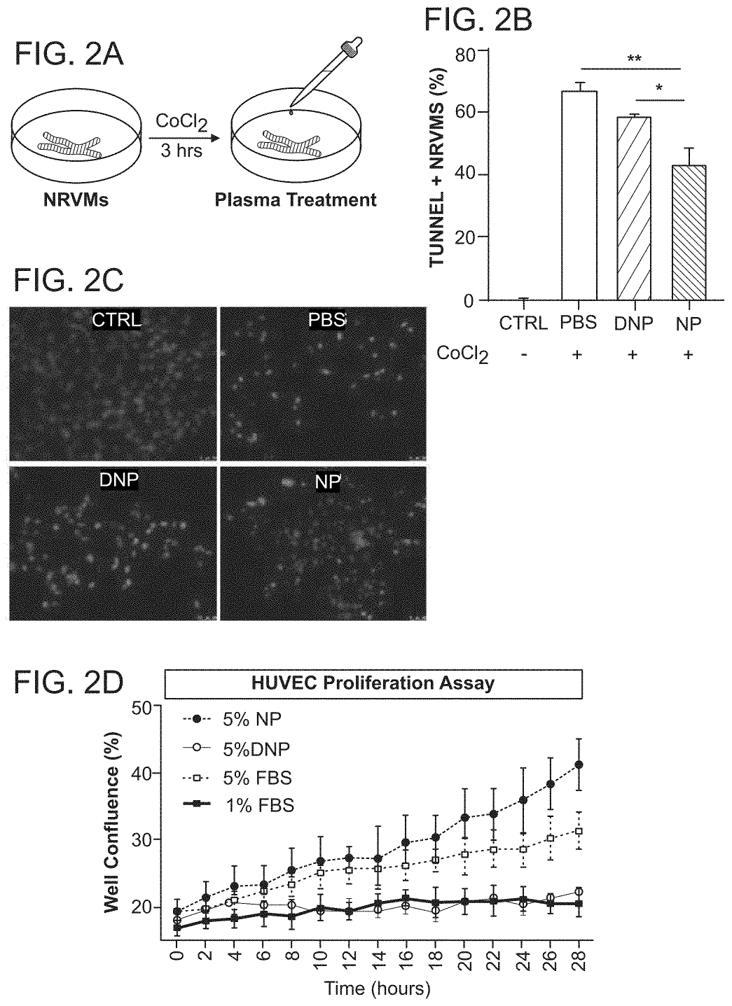

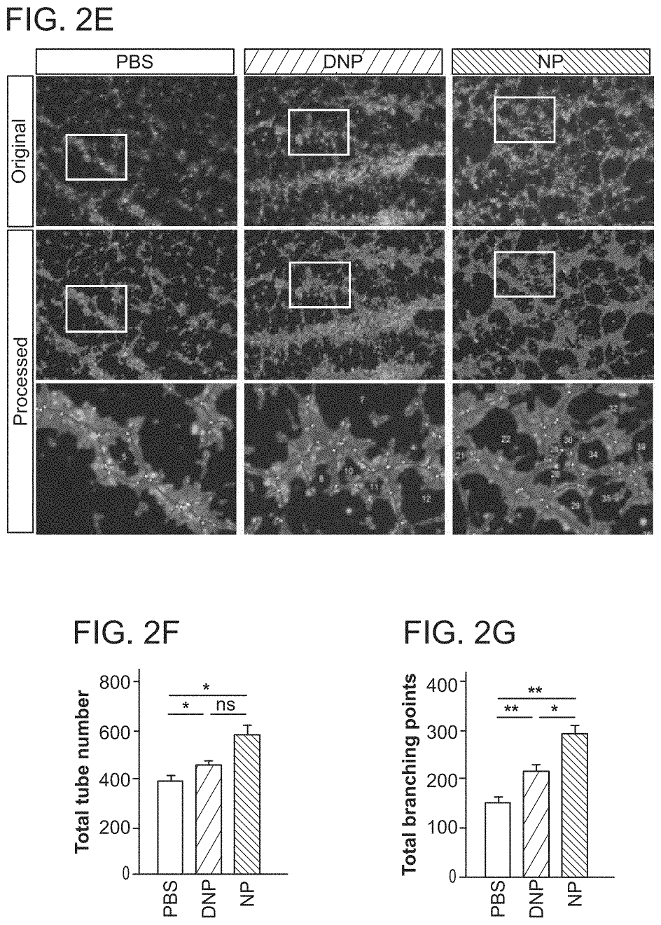

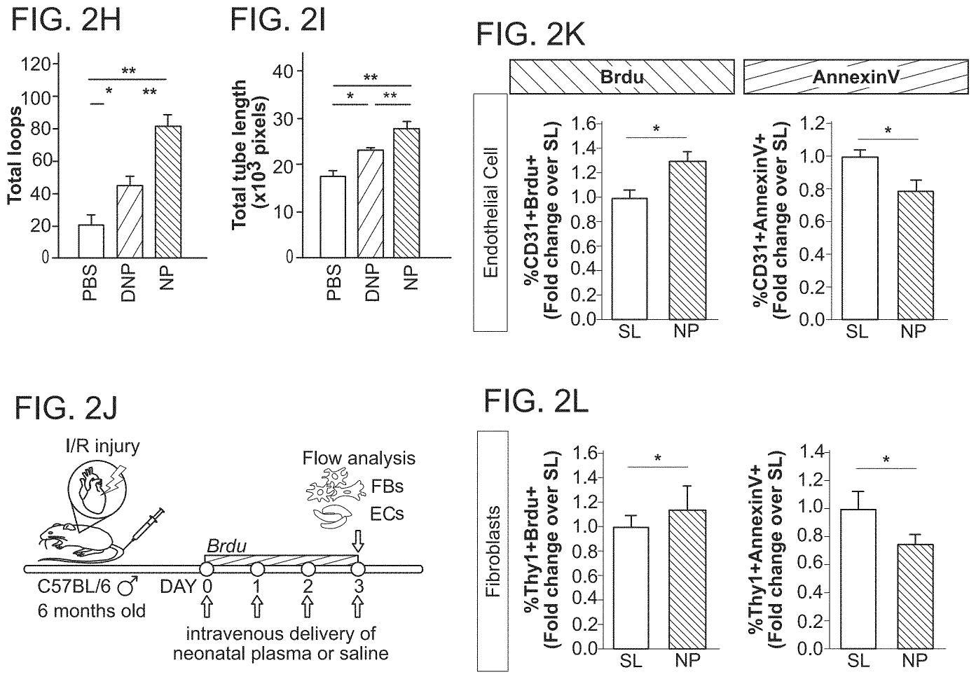

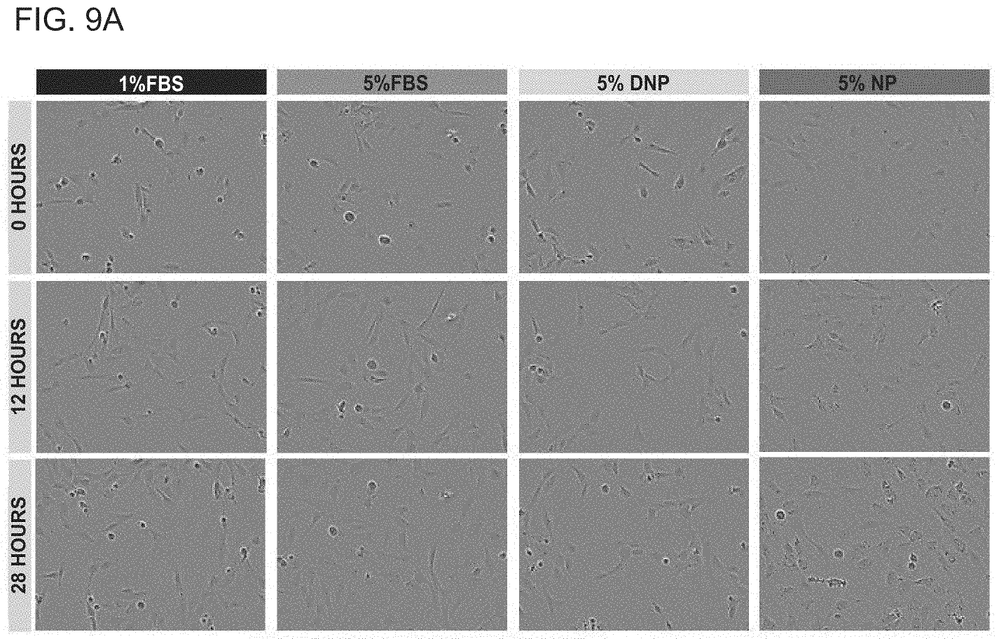

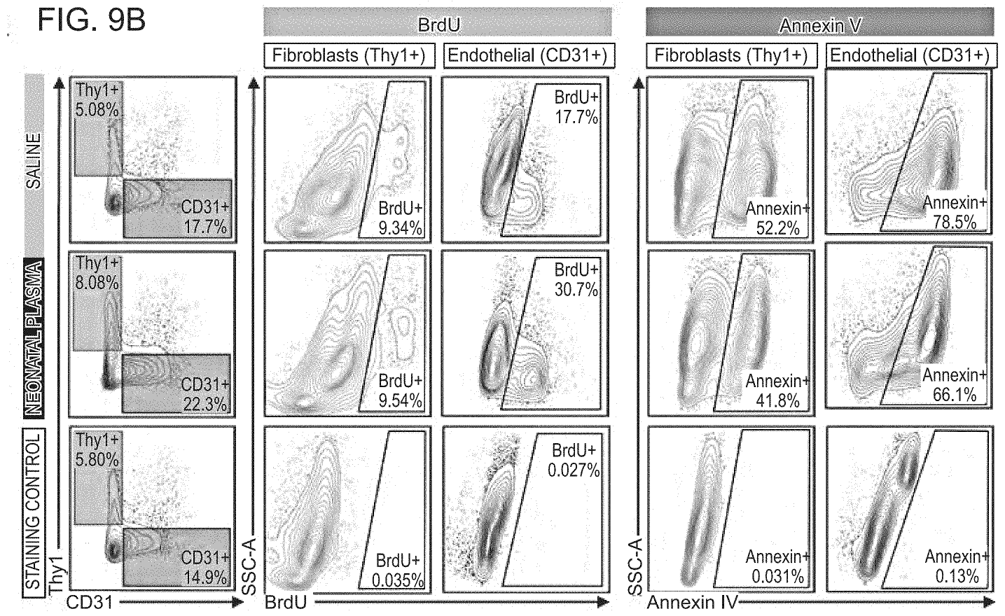

As systemic factors have been previously shown to mediate a wide variety of effects in different cell types, the study examined whether the improvement in cardiac function and scar size can be attributed to specific responses within the major cardiac cell types, namely cardiomyocytes, fibroblasts, and endothelial cells. The study first examined whether neonatal plasma affects the extent of hypoxia-induced apoptosis in neonatal rat ventricular myocytes (NRVMs). NRVMs were stressed with cobalt chloride for 3 hours and then switched to normal growth media supplemented with plasma for 24 hours (FIG. 2a). TUNEL staining revealed a significant decrease in the percent of TUNEL+ cells with neonatal plasma treatment compared to controls (FIG. 2b,c). The study then examined the effect of neonatal plasma on proliferation and tubal formation capability of human umbilical vein endothelial cells (HUVECs). Quantification of well confluence (as an indirect measure of proliferation) over 28 hours showed that neonatal plasma increased the rate of proliferation compared to FBS controls and interesting, this effect was not observed in denatured neonatal plasma (FIG. 2d and FIG. 9a). Similarly, neonatal plasma treatment improved various metrics of tubal formation compared to PBS and denatured neonatal plasma in an in vitro angiogenesis assay (FIG. 2e-i). The study also examined proliferation and apoptosis of endothelial cells and fibroblasts in vivo. C57BL/6 adult mice were given 4 doses of neonatal plasma after I/R injury and the extent of BrdU and Annexin V labeling were determined with flow cytometry (FIG. 2j). Within endothelial cells, neonatal plasma treatment significantly increased the percent of BrdU+ cells, with a concomitant decrease in the percent of Annexin V+ labeling (FIG. 2k and FIG. 9b). Fibroblasts, determined as Thy1+ cells, showed similar trends although were not significant compared to controls (FIG. 2I and FIG. 9b).

### Single Cell RNA Sequencing Highlights an Anti-Apoptosis Role of Neonatal Plasma

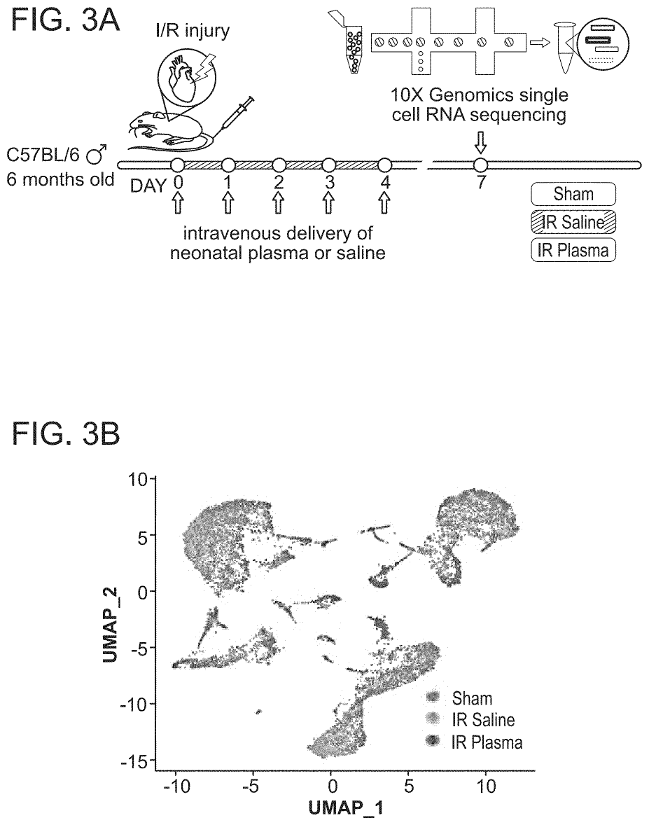

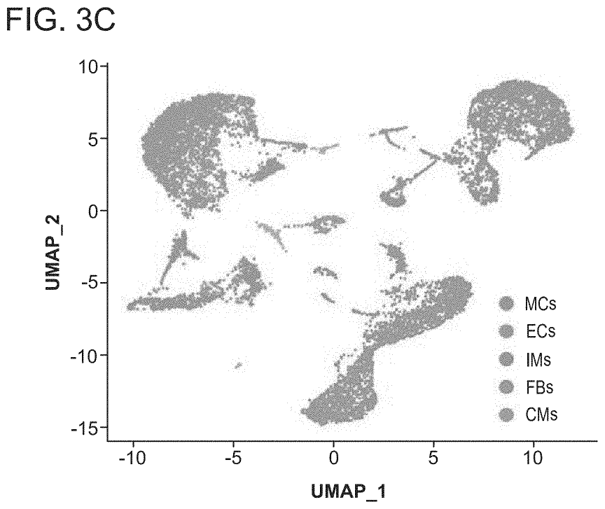

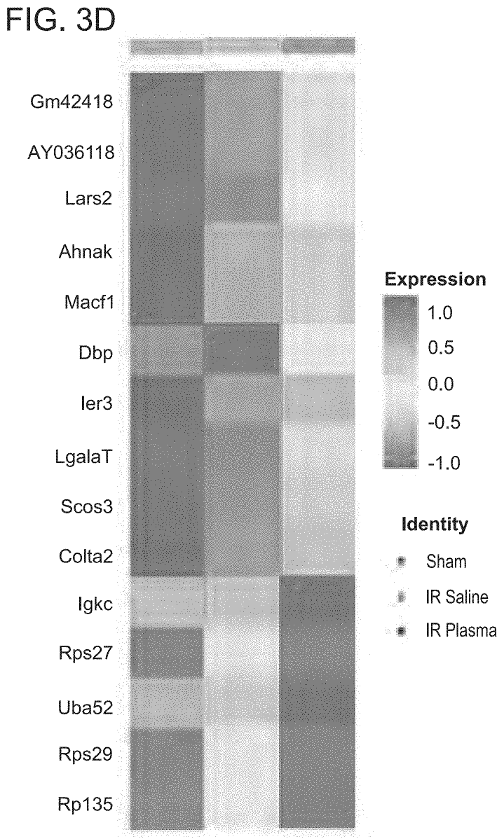

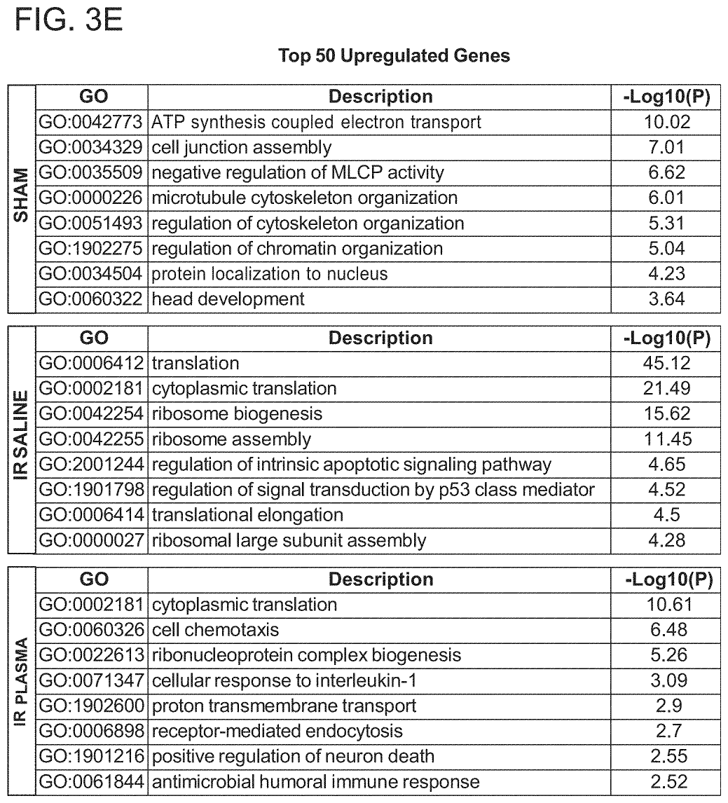

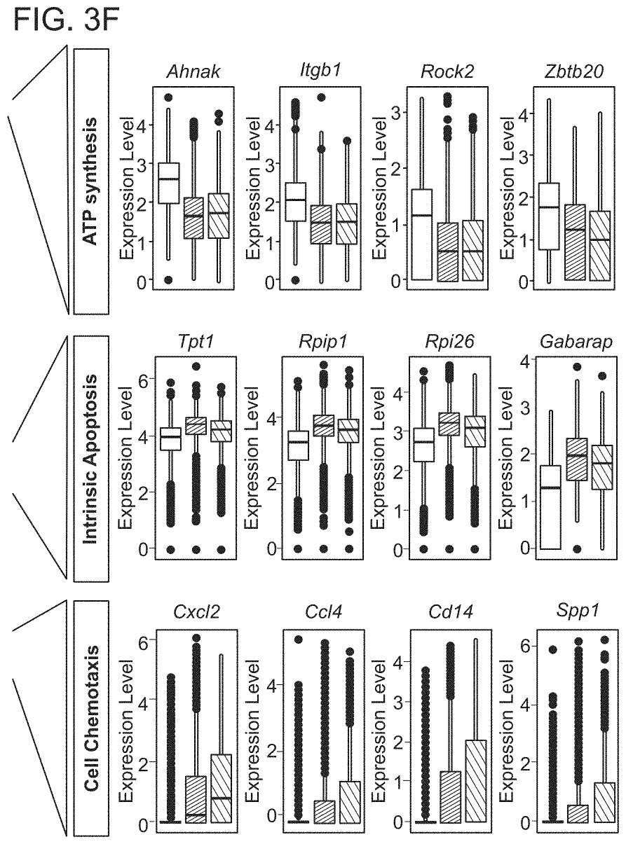

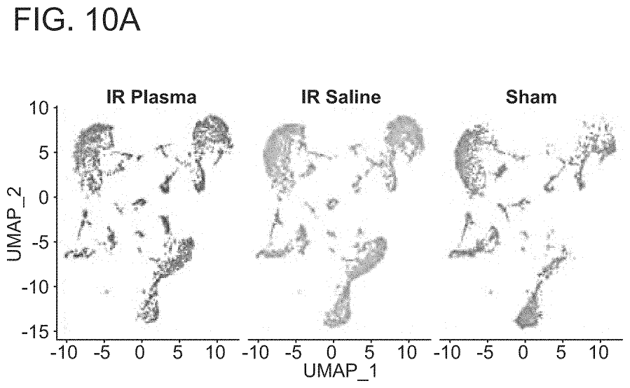

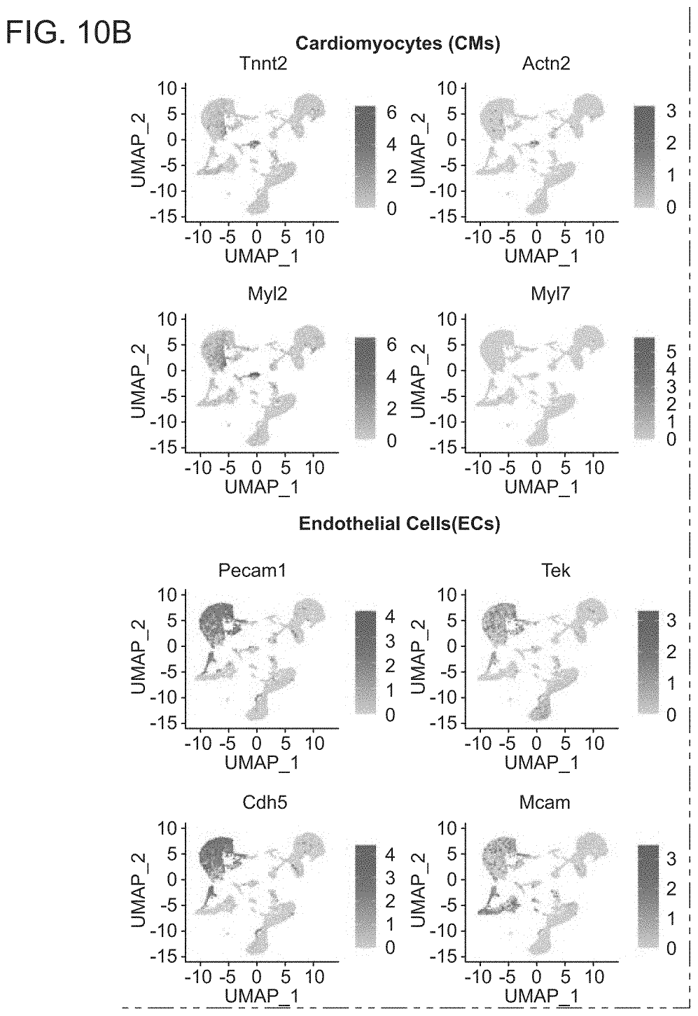

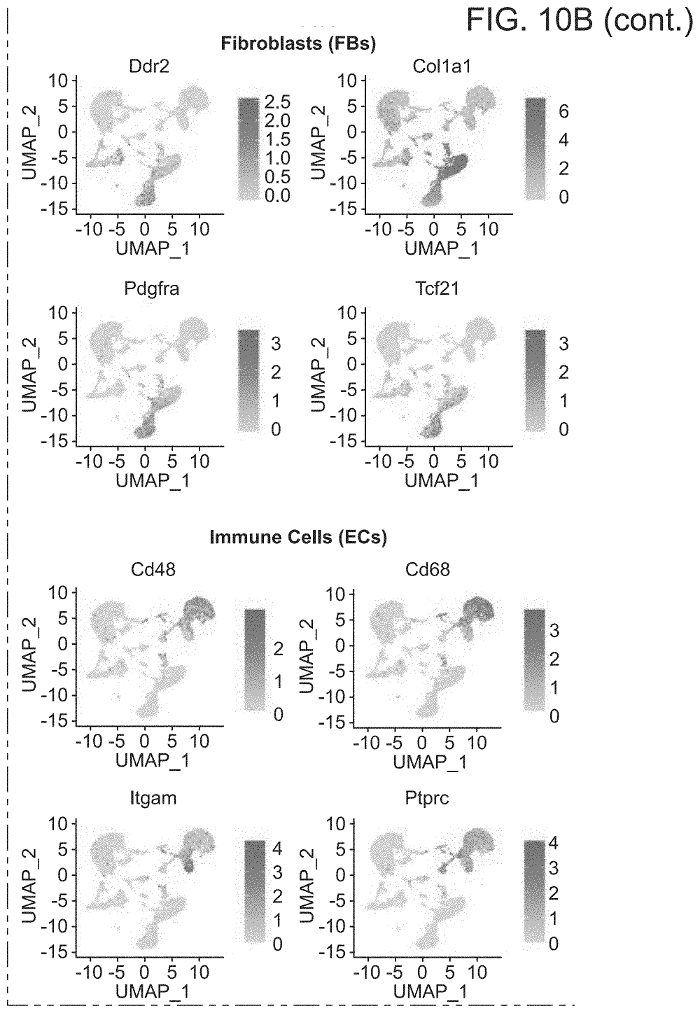

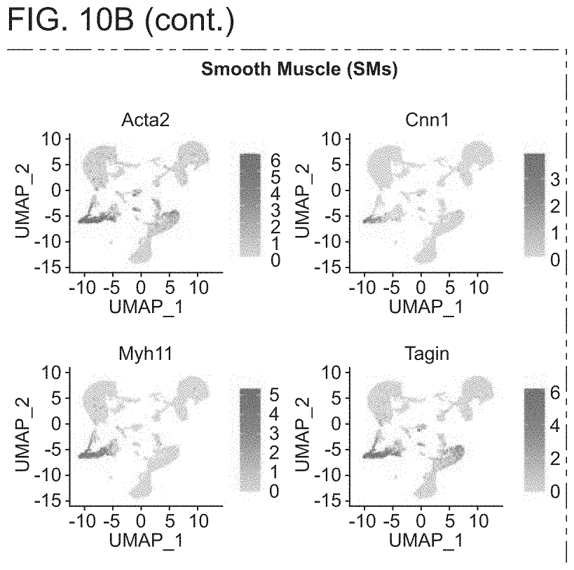

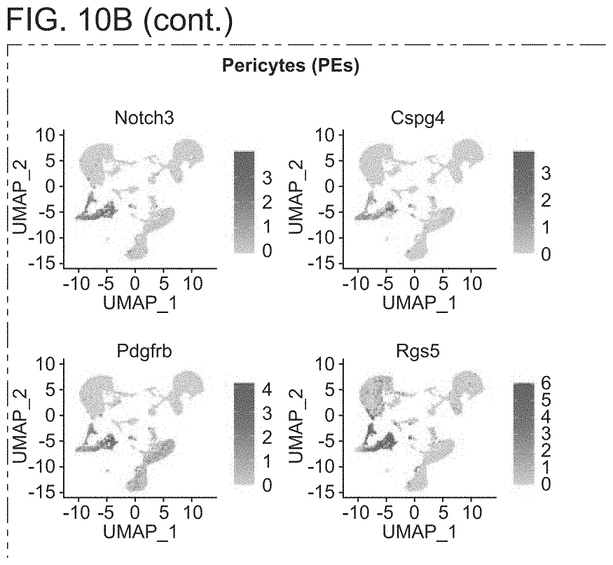

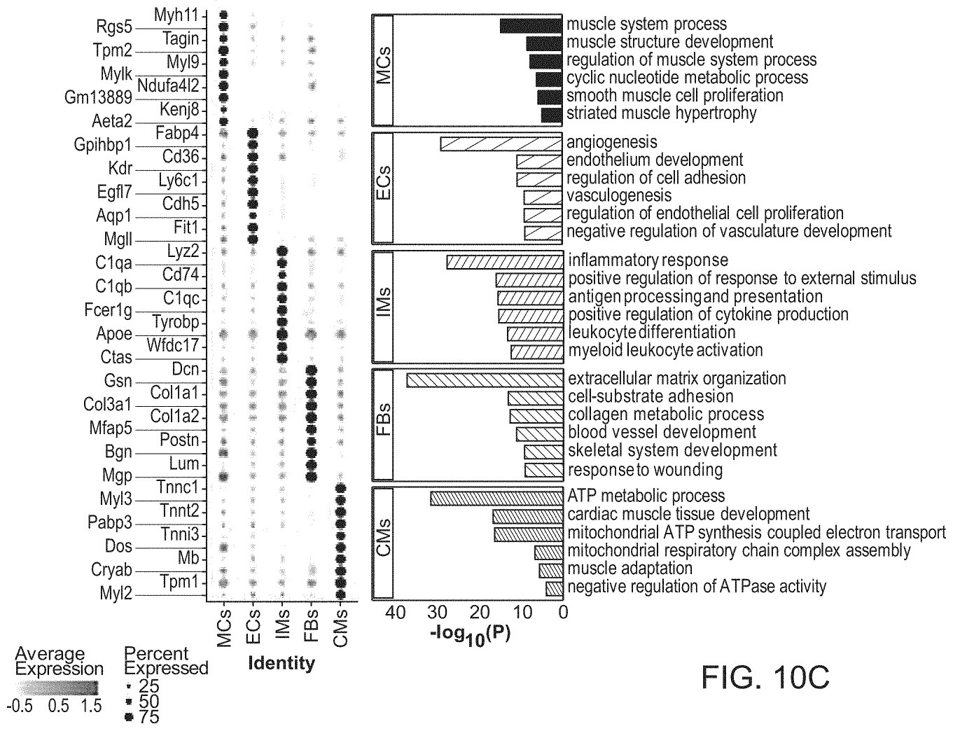

Given both the phenotypic and cellular changes observed in response to neonatal plasma treatment, the study next sought to examine its effect at the transcriptomic level. To do so, the study turned to single cell RNA sequencing (scRNA seq) as a platform that would allow for profiling of the whole heart at the level of individual cells. Male C57BL/6 mice 6 months of age were subjected to experimental I/R injury followed by intravenous delivery of neonatal plasma or saline control for 4 consecutive days (FIG. 3a). Sham animals served as surgery control. On Day 7 after injury, single cells digested from the left ventricles of hearts within each experimental group were pooled and captured for RNA sequencing using the 10× Genomics platform. In total, 3,250 (Sham), 5,118 (IR Saline), and 2,754 (IR Plasma) passed quality control processing. UMAP analysis shows a general overlap of cells from different treatment groups (FIG. 3b and FIG. 10a). By plotting expression of established cardiac cell type markers, the study were able to identify distinct clusters as endothelial cells (ECs), immune cells (IMs), fibroblasts (FBs), cardiomyocytes (CMs), and combined smooth muscle and pericytes (MCs) (FIG. 3c and FIG. 10b). GO analysis of the top 100 genes within each of the cell clusters confirmed their identity (FIG. 10c).

The study first looked at differential gene expression between the different treatment groups to determine whether the transcriptomic profile of hearts treated with neonatal plasma differs from saline control. As expected, the profile of sham control, in which the LAD was not ligated, was distinct from both the saline and neonatal plasma treated groups (FIG. 3d-f) and GO biological process of the top 50 genes enriched in this group revealed pathways associated with normal cardiac function such as “ATP synthesis coupled electron transport” and “cell junction assembly” (FIG. 3e). In both saline and neonatal plasma treated groups, processes such as “cytoplasmic translation” and “ribosome biogenesis” were enriched, likely attributed to the ischemia-reperfusion injury. Interestingly, apoptosis-related pathways were observed in saline treated groups whereas cell chemotaxis and immune-related pathways were enriched in neonatal plasma treated group (FIG. 3e-f).

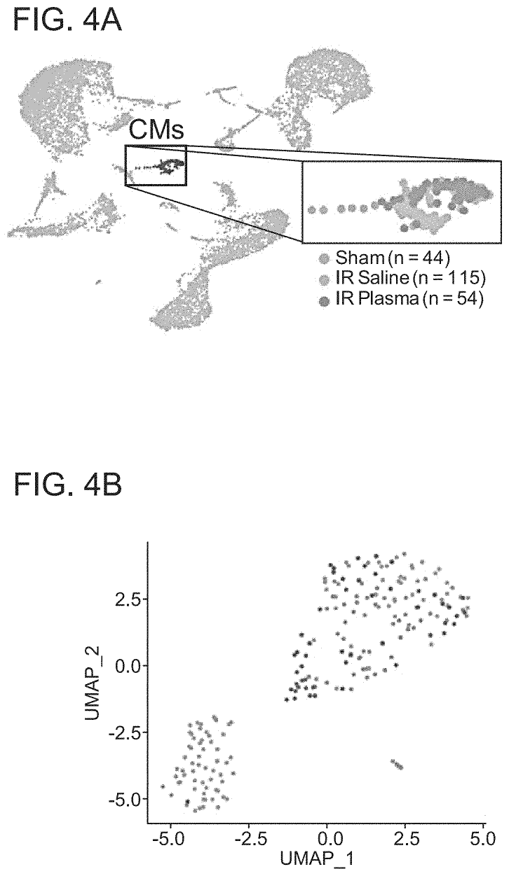

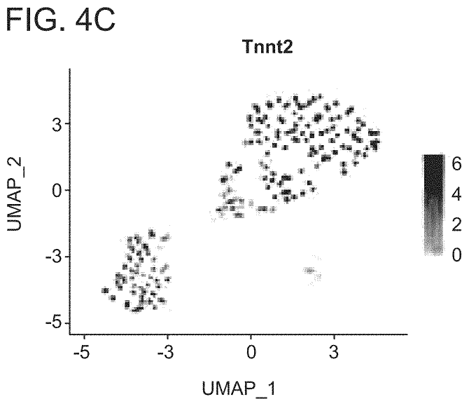

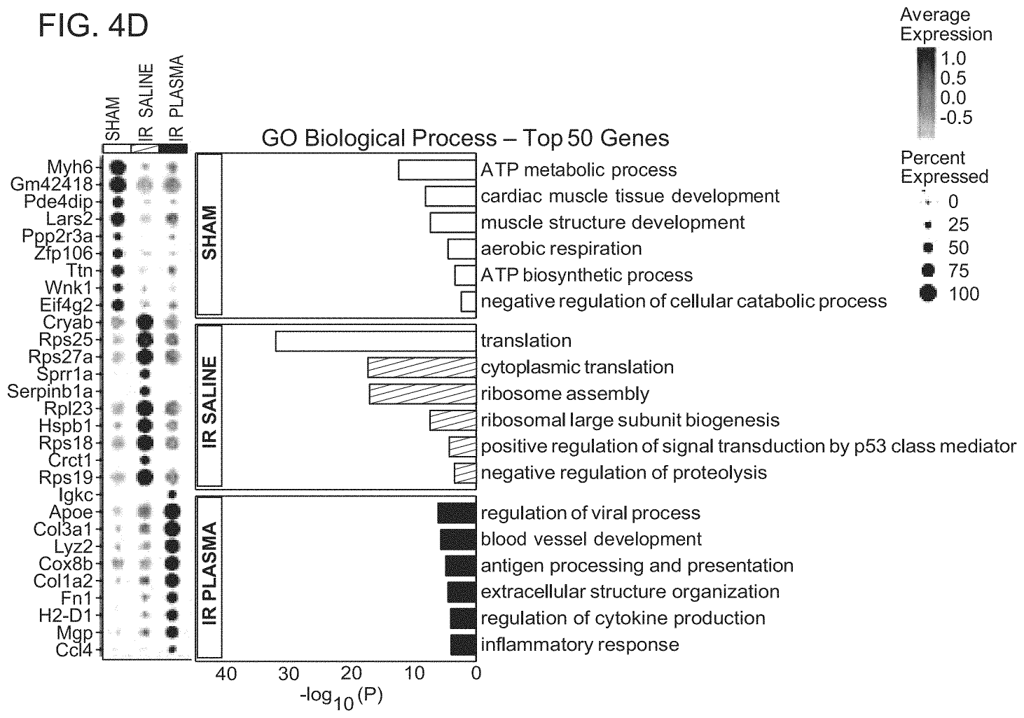

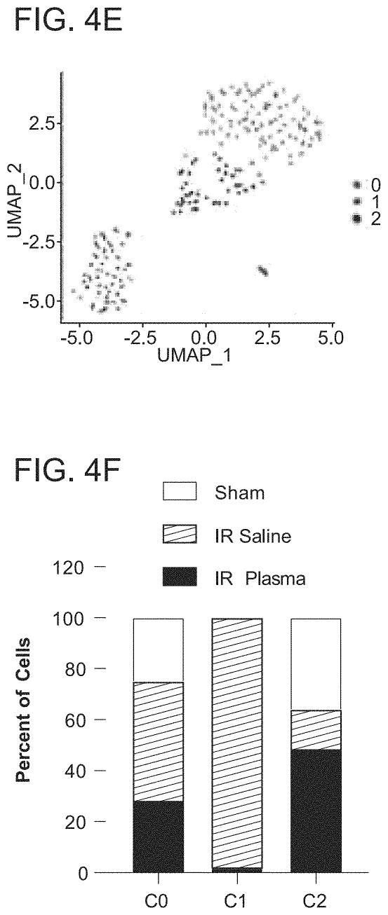

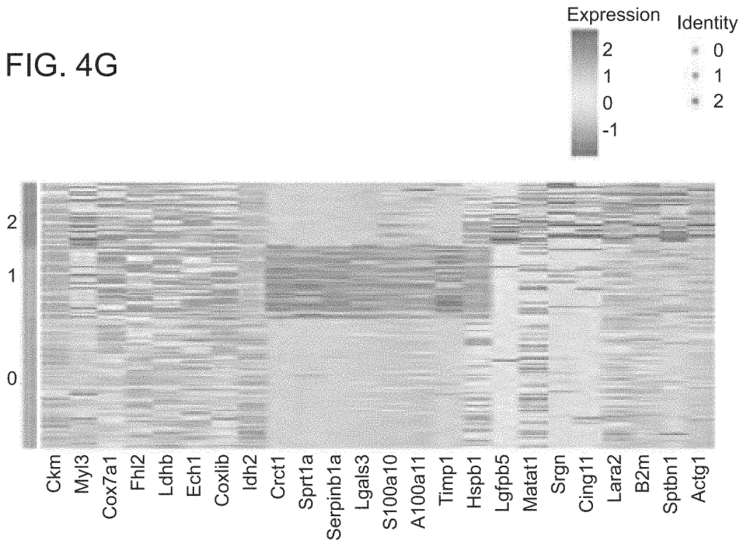

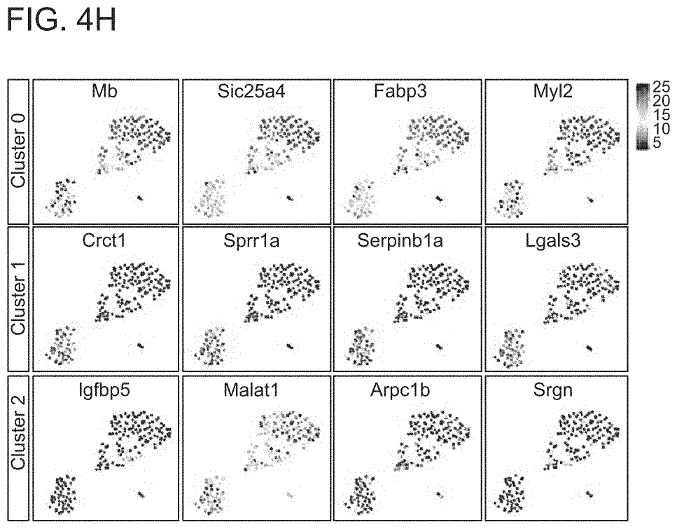

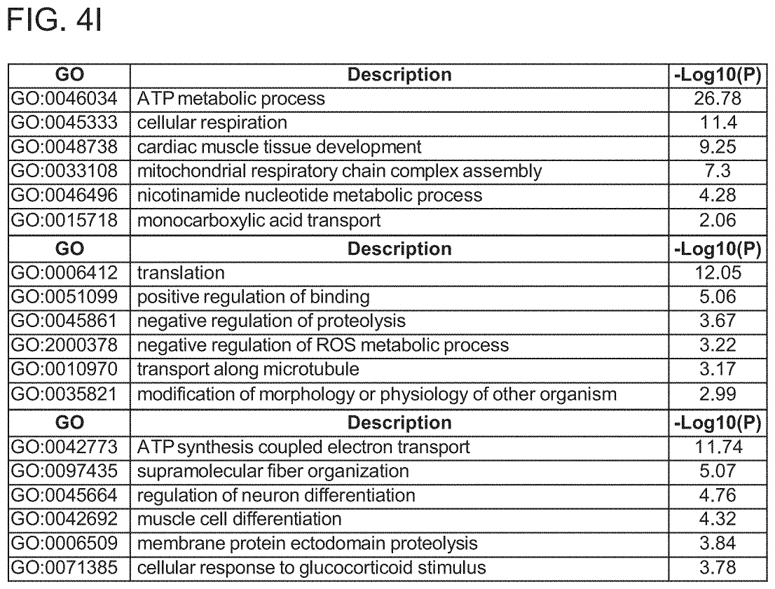

To examine cell-type specific responses to neonatal plasma administration, CMs were subsetted for further analysis (FIG. 4a-c). UMAP dimension reduction shows a visually distinct cluster of CMs composed predominantly of cells from the saline group (FIG. 4b). Differential gene expression and pathway analysis revealed that CMs from the saline group expressed more genes associated with translation and apoptosis pathways whereas neonatal plasma promoted more immune-related pathways (FIG. 4d). As the location of cells relative to the infarct region may result in differences in severity of ischemic injury (i.e., cells closer to the ligated region may experience more severe ischemia compared to remote regions), it is possible that these CM clusters identified may be separated by extent of injury, in which the study can expect CMs with less severe ischemia to cluster with cells from the sham control. To investigate this, the study performed clustering analysis of CMs which showed three distinct clusters of cells, of which Cluster 1 is predominantly composed of CMs from saline group and Cluster 2 from neonatal plasma group (FIG. 4e,f). Differential gene expression and pathway analysis shows an enrichment of Cluster 0 in pathways such as ATP metabolic process and cellular respiration, suggestive of normal CM function (FIG. 4g-i). Cluster 1, containing the majority of CMs from the saline group, exhibited pathways relating to translation and ribosome biogenesis whereas Cluster 2, containing a majority of plasma treated CMs, were enriched in ATP synthesis coupled electron transport and muscle cell differentiation, suggestive of their shift toward normal CM function (Cluster 0), which is also visually observed on UMAP (FIG. 4e). Of note, the study identified several genes (Crct1, Sprr1a, Serpinb1a, Lgals3) whose expression was mostly specific to only Cluster 1 (FIG. 4h). Together, the scRNA seq profiling shows distinct gene expression changes with neonatal plasma administration, indicating their effect at the transcriptomic level.

### Mass Spectrometry Reveals Differences in Associated Pathways and Molecular Functions of Differentially Abundant Proteins Between Neonatal and Adult Mouse Plasma

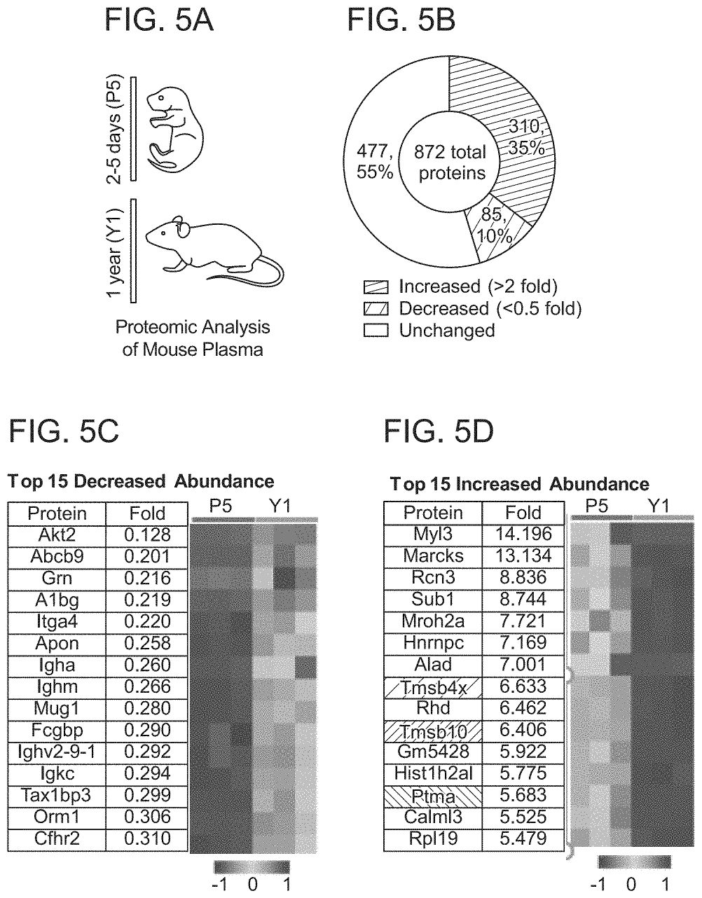

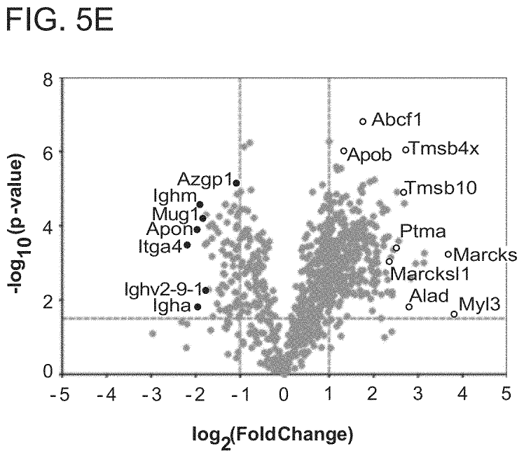

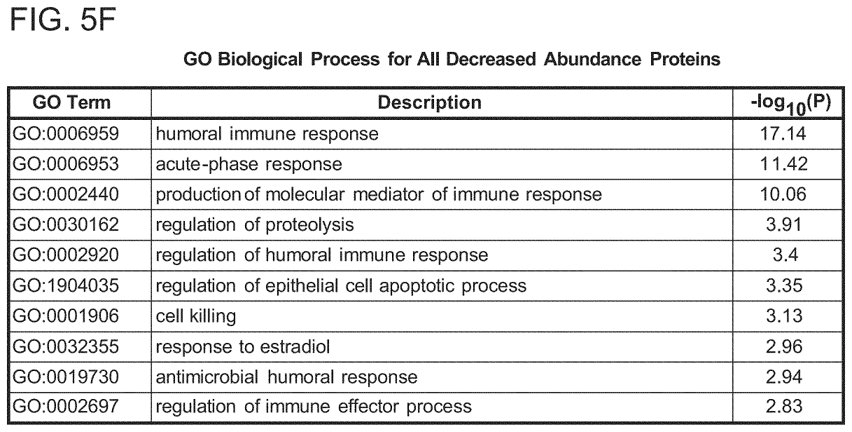

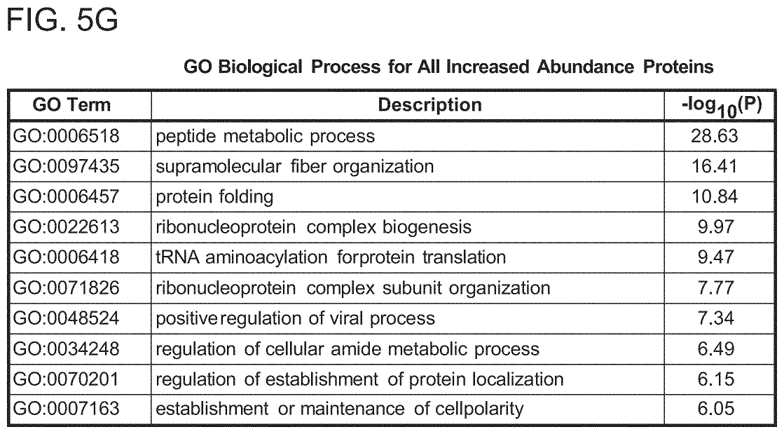

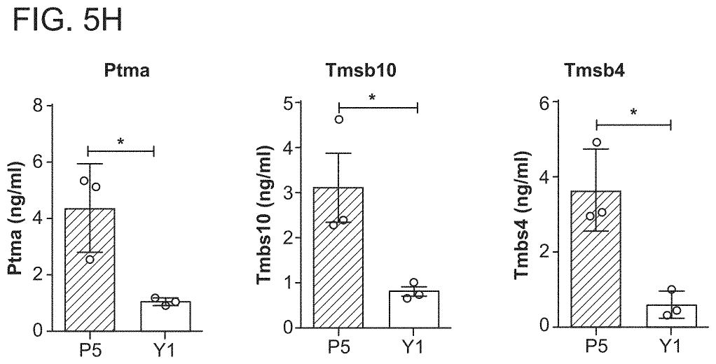

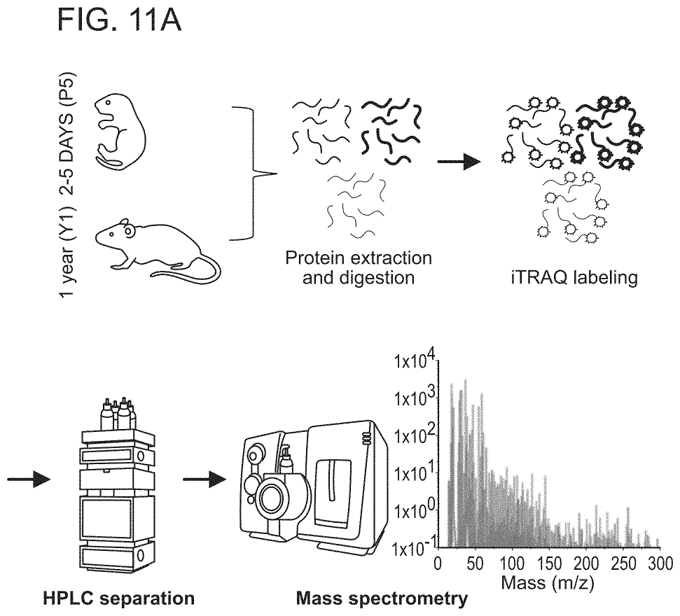

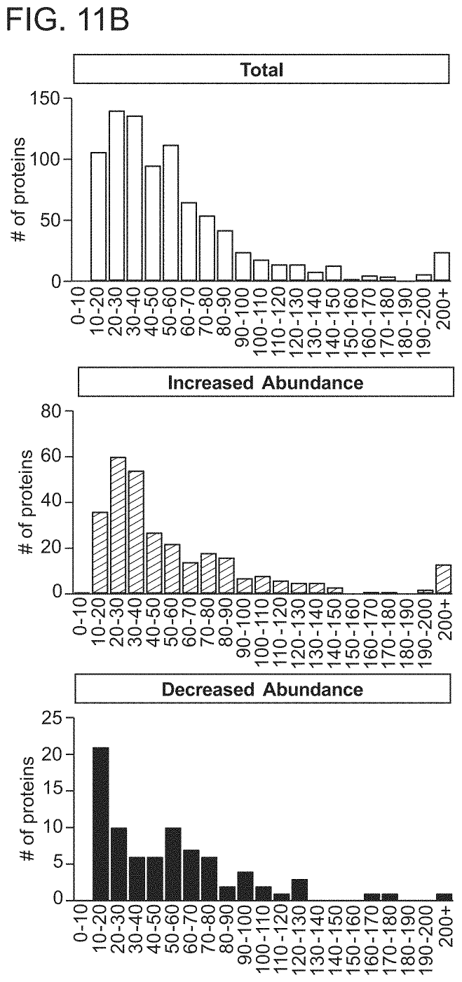

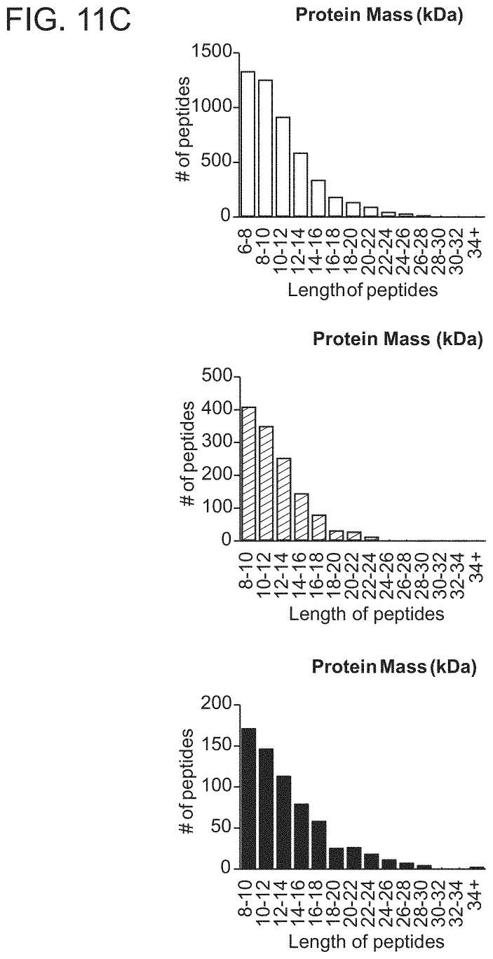

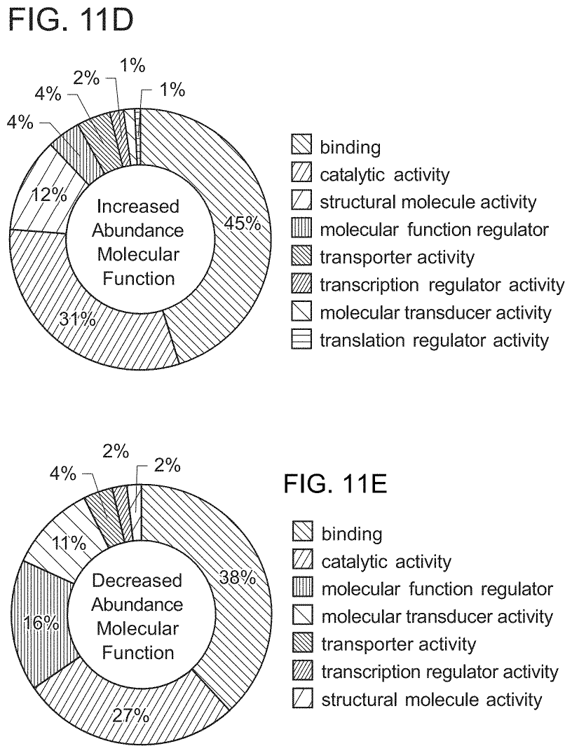

To identify candidates that may mediate both the functional and transcriptomic changes observed, the study performed quantitative mass spectrometry on plasma from neonatal and aged mice using the isobaric tag for relative and absolute quantitation (iTRAQ) methodology (FIG. 5a and FIG. 11a). In brief, proteins were extracted from plasma samples and high abundance proteins were depleted (immunoglobulins and albumin). Samples were then digested, labeled with iTRAQ reagents, and fractionated for mass spectrometry. Database analysis revealed a total of 872 proteins, of which 310 and 85 were determined to be of increased (>2.0) and decreased (<0.5) abundance, respectively, within neonatal plasma compared to aged plasma (FIG. 5b-e). Biological pathway analysis of all decreased abundance proteins shows enrichment of immune-related processes whereas increased abundance proteins show a mixture of metabolic and ribonucleoprotein biogenesis (FIG. 5f,g). Protein mass and peptide length histograms demonstrate low molecular weights and lengths of identified proteins, with “binding” as the key molecular function (FIG. 11b-e).

### Thymosin Proteins are Enriched in Neonatal Plasma

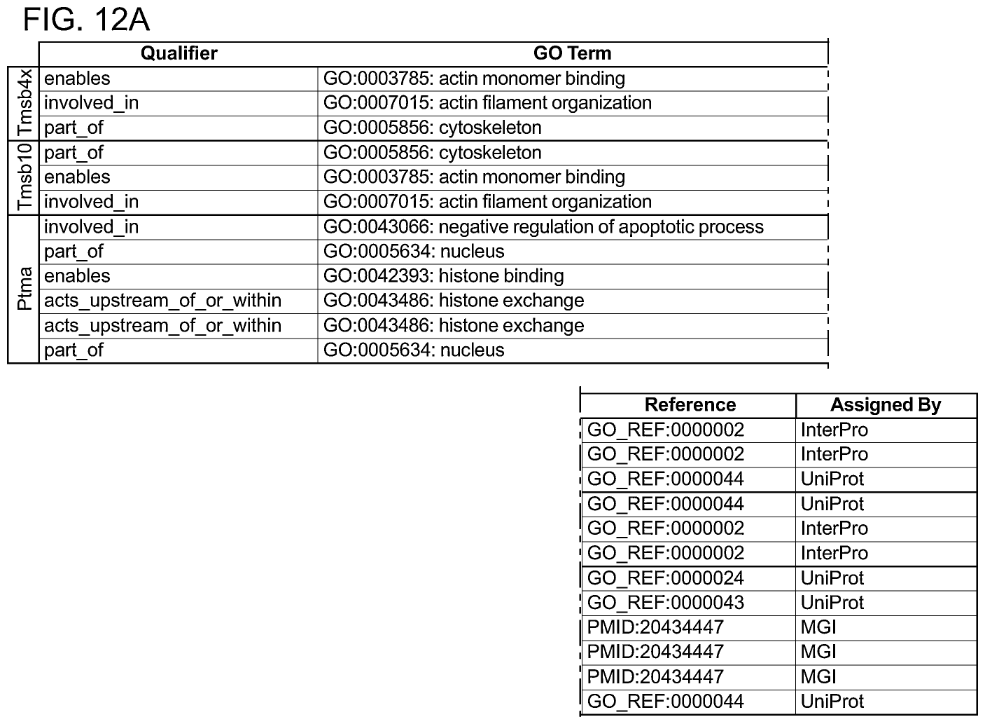

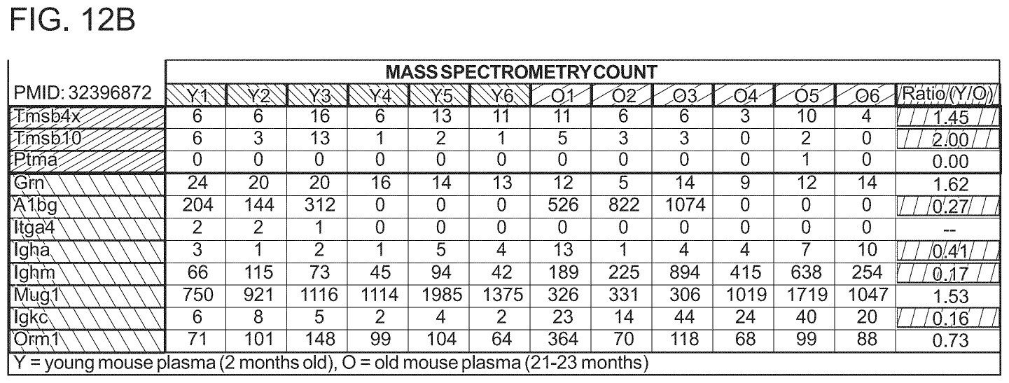

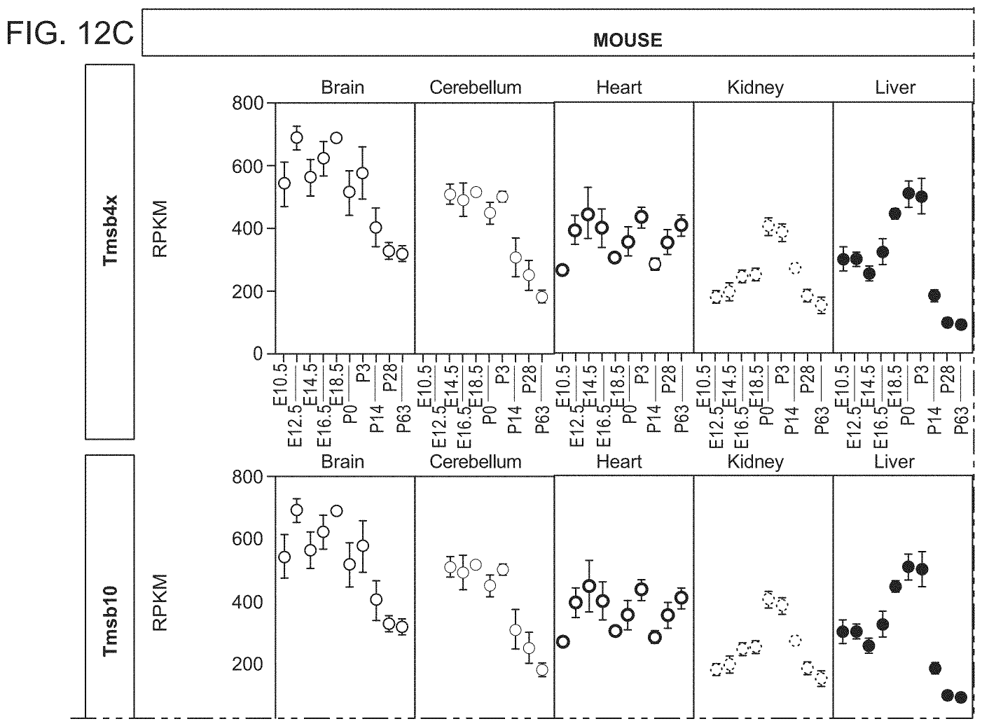

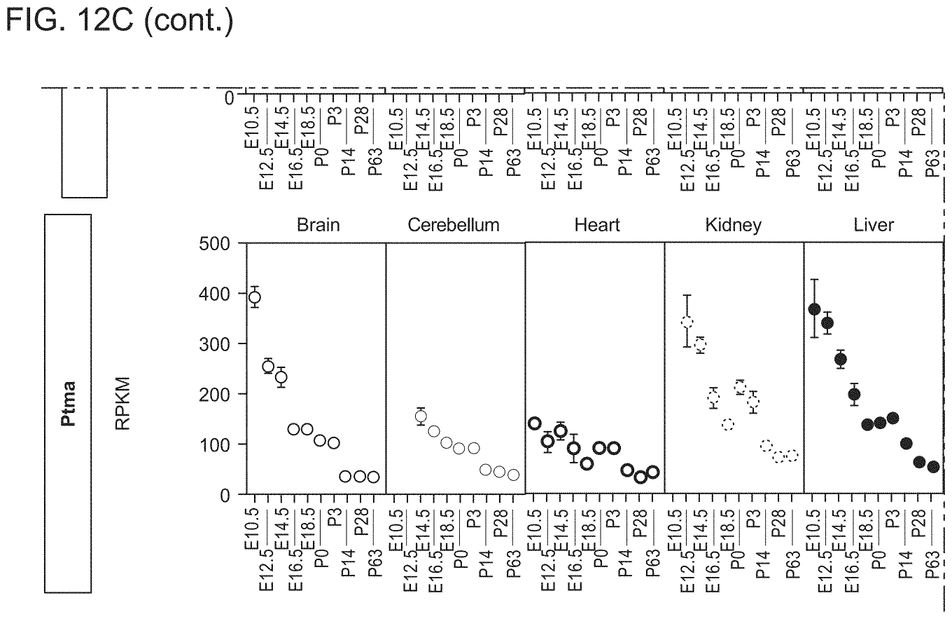

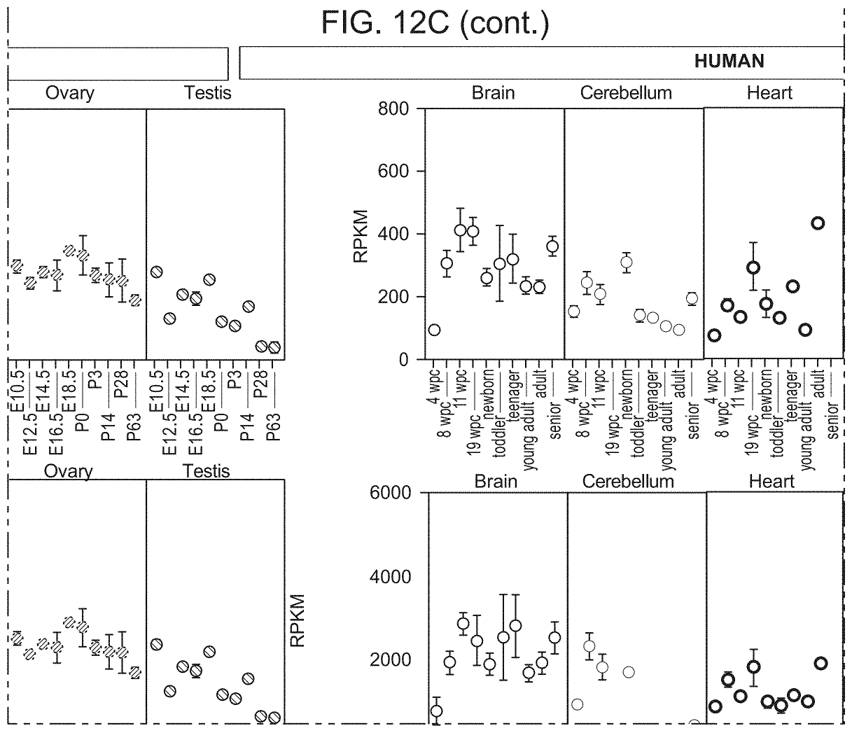

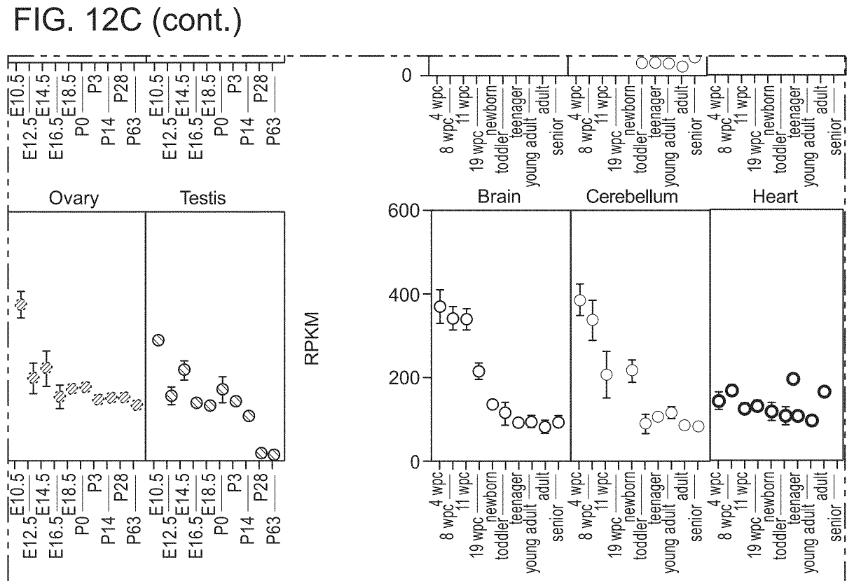

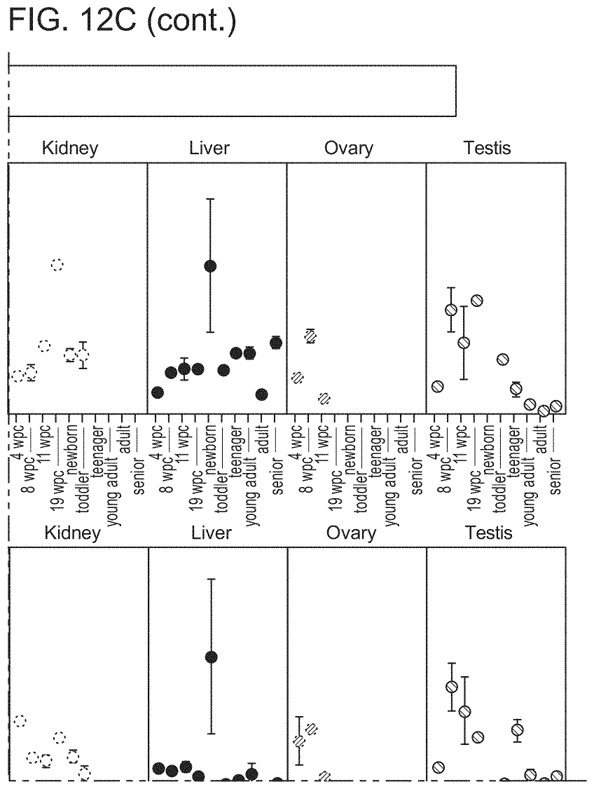

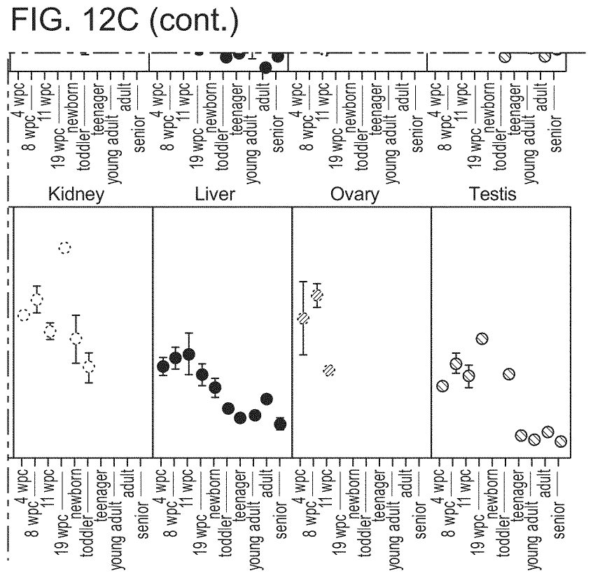

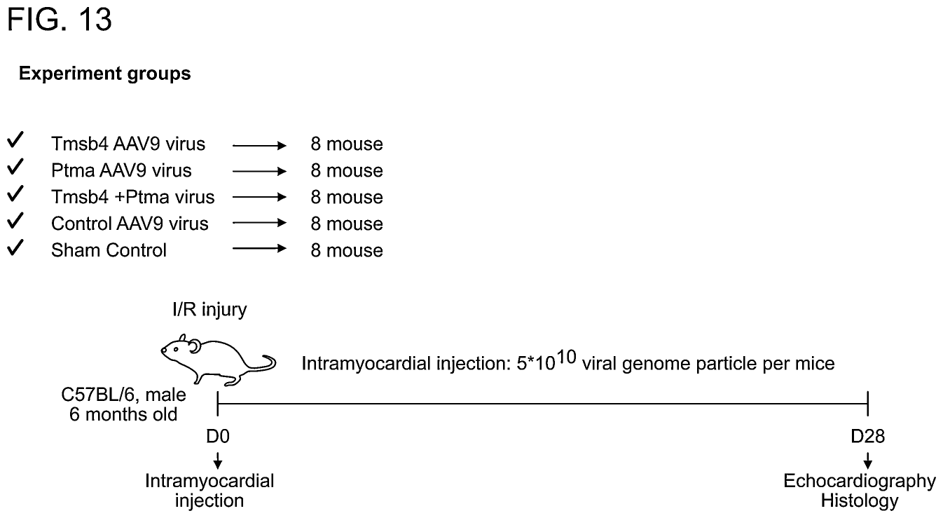

Of the top candidates identified from mass spectrometry, three increased abundance proteins were from the thymosin family: Tmsb4x (Thymosin beta 4), Tmsb10 (Thymosin beta 10), and Ptma (Prothymosin alpha) (highlighted in FIG. 5d). Tmsb4x and Tmsb10 are associated with GO terms relating to actin binding and organization whereas Ptma has been shown to be involved in apoptosis and histone binding/exchange (FIG. 12a). Both Tmsb4x and Tmsb10 were previously identified to be of increased abundance in young compared to aged plasma²⁵ (FIG. 12b). To further assess the potential of these candidates as age-dependent factors that may be mediating the beneficial effects observed, the study used the Kaessmann database²⁶, containing RNA sequencing results of various organs in 7 different species spanning across developmental age. Interestingly, the study observed an overall decline with age in the expression of these three candidate genes across different organs, with the most consistent trend being Ptma expression (FIG. 12c).

Thymosin Proteins Protect Cardiomyocytes from Ischemia-Induced Apoptosis

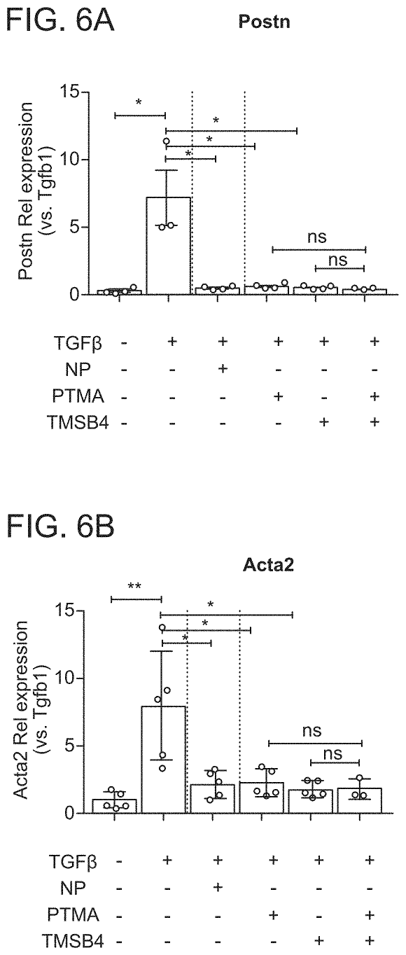

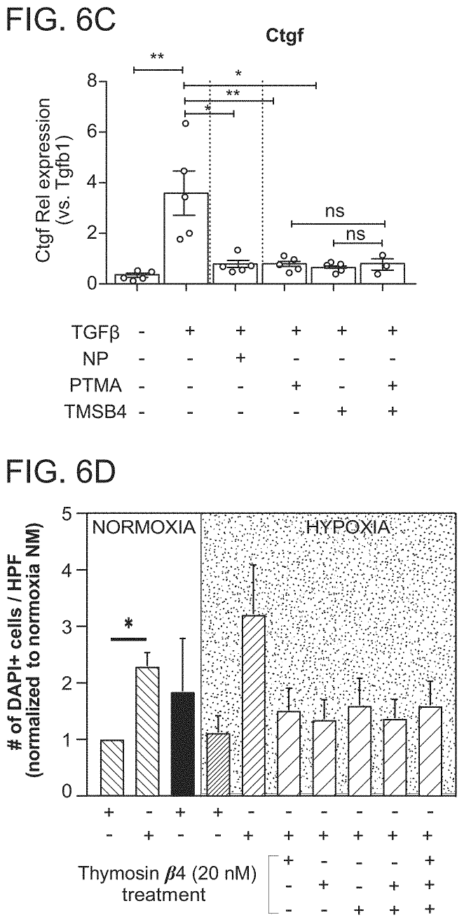

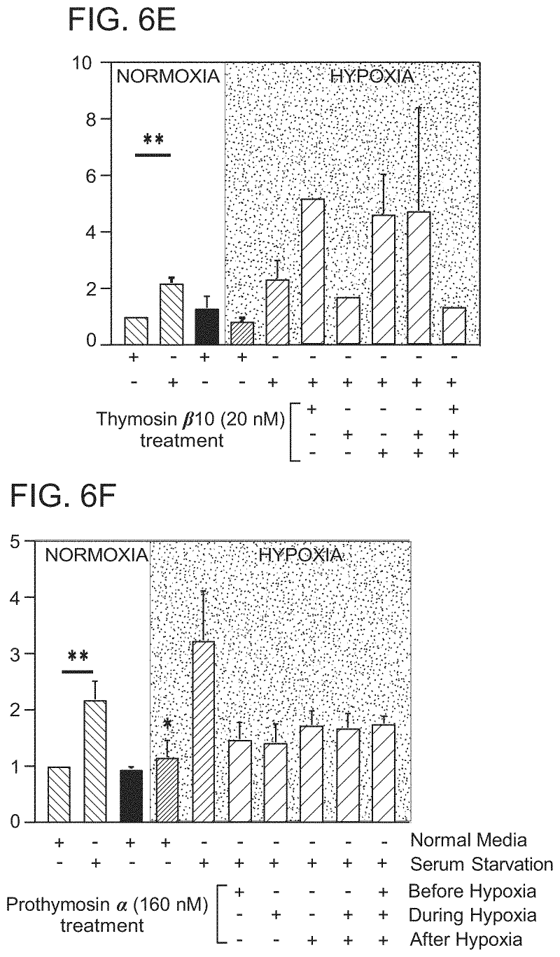

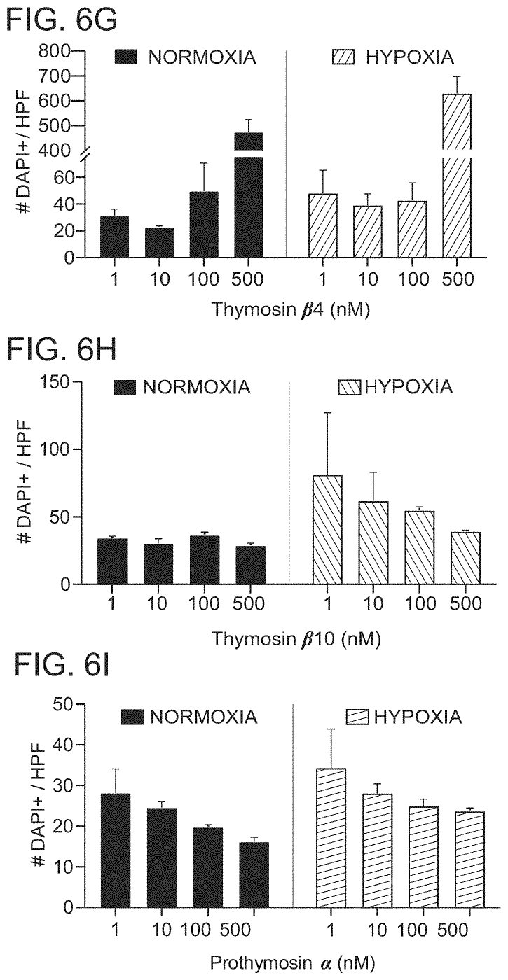

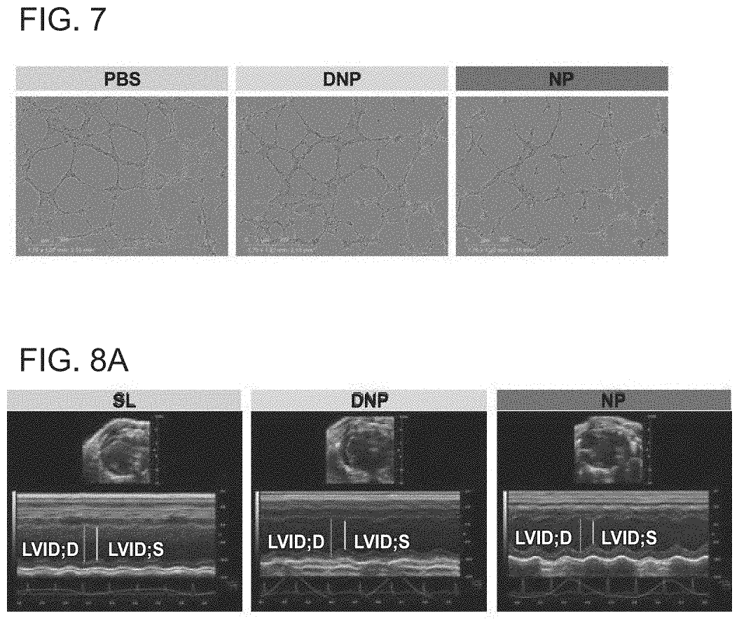

The cross-examination of the identified candidates with online databases and previously published studies likely demonstrates the age-dependence of these three thymosin proteins. To examine potential functional roles of these candidates, particularly on cellular apoptosis, the study next used an in vitro hypoxia assay of HL-1 CMs, in which cells were exposed to low oxygen (1%02) for 6 hours prior to treatment with recombinant forms of these proteins. Quantification of the number of DAPI+ cells per field showed that treatment with either thymosin β4 or prothymosin α reduced the number of DAN+HL-1 CMs (FIG. 6d,f). This effect is not consistently observed with thymosin β10 although a dose-dependent effect was present (FIG. 6e,h). While thymosin β4 displayed a protective effect, the study observe a small therapeutic window, as high doses (500 nM) resulted in cellular toxicity (FIG. 6g). This is in contrast to prothymosin α, in which high concentrations did not have a negative effect on cellular viability (FIG. 6i). Quantification of both EdU incorporation and pHH3 immunoreactivity showed no effect of these three candidates on cellular proliferation (data not shown).

### Discussion

The search of “pro-youthful” factors to delay the aging process has been an elusive quest. Researchers have turned to parabiosis as a way to examine effects of shared circulation and to identify potential factors as a regenerative therapy. The study aimed to understand the role of systemic factors in the context of cardiac ischemia-reperfusion injury. Given the known ability of the neonatal heart to fully regenerate after injury, it was hypothesized that neonatal circulation may be more enriched with factors that promote repair and regeneration. The in vivo study showing improved cardiac function and reduced scar sizes in hearts of mice after ischemia reperfusion injury implicates a protective effect of neonatal plasma. At a cellular level, the study find that neonatal plasma ameliorates apoptosis of cardiomyocytes and promotes tubal formation of endothelial cells, both of which are key processes involved in cardiac regeneration. A decrease in the degree of cardiomyocyte apoptosis may shift cells toward autophagy during the early and critical phases of remodeling to minimize cellular loss and maintain cardiac contractile function.

The finding of diminished biological effects via the denaturing process recapitulates the significance of proteins from prior studies and led the study to examine the proteomic profile of neonatal and aged mouse plasma. The generated dataset provides a unique look into the proteome during a period of development that is much earlier than prior studies. Of the candidates identified, the study were particularly interested in prothymosin α, thymosin β4, and thymosin β10, given their name association with the thymus, an organ whose size and function changes with developmental age, as well as the known roles of thymosin β4 in cardiac repair. The thymus plays a key role in the development of T cells during fetal and early development and is, therefore, vital for proper function of the adaptive immune response. However, their function is dispensable after puberty leading to involution of the organ. The increased abundance of these thymosin proteins within neonatal plasma reflects the known function and development of this immune organ. The in vitro finding of a protective role of both prothymosin alpha and thymosin β4 in cardiomyocyte apoptosis suggests of their therapeutic potential. It has been previously shown that the anti-apoptotic function of Ptma may be regulated via the Akt signaling pathway, which itself is associated with a multitude of cellular processes such as cellular proliferation and growth. Interestingly, the initial analysis of the transcriptomic profile of cardiomyocytes shows an enrichment of apoptosis-associated pathways in saline control whereas neonatal plasma administration shifts the biological processes toward more immune-related terms.

While the study have primarily focused on the existence of “pro-youthful” factors within neonatal plasma, it is worthy to note an alternative therapeutic strategy, one that is focused on the identification and inhibition of “pro-aging” factors that may play a role in the injury response. The proteomic profile highlights a decrease in immune-related factors within neonatal compared to aged plasma, which is in alignment with the increased presence of thymic proteins. Further studies are warranted to dive deeper into this aspect of cardiac repair. Furthermore, while the study observe a protective role of both prothymosin α and thymosin β4, it may be likely that their combined treatment may offer greater protection from cardiomyocyte apoptosis. In summary, the study highlights a protective effect of neonatal plasma and the potential role of thymosin proteins in mediating cardiac repair. It brings to light the potential of neonatal plasma as a rejuvenative therapy for cardiovascular as well as other age-related diseases.

### Methods

### Neonatal and Adult Mouse Plasma Collection

Neonatal mice (postnatal days 2-5) were anesthetized on ice for 2 minutes. Aged mice were anesthetized using a 3% isoflurane chamber. A thoracotomy was then performed to reveal the heart for cardiac puncture blood collection into a K2EDTA-microtainer (BD, 365974). Blood samples were centrifuged 10 min at 1000×g and supernatant (plasma portion) collected and stored at −80° C. until use.

### Ischemia-Reperfusion Injury and Plasma Administration

Ischemia-reperfusion injury was induced via permanent ligation of the left anterior descending artery (LAD). C57BL/6 mice six months of age were anesthetized by intraperitoneal injection of ketamine (100 mg/kg) and xylazine (10 mg/kg). A left thoracotomy was performed through an incision between the fourth and fifth intercostal muscles followed by removal of the pericardium. An 8-0 silk suture was used to temporarily ligate the LAD and released after 45 minutes. Post-operative discomfort was treated with buprenorphine (0.03-0.06 mg/kg). Sham-operated mice were submitted to the same procedure lacking the LAD ligation. Plasma (200 μl each dose) was administered via the tail vein on day of surgery and daily for designated subsequent number of days. All animal studies were performed according to the guidelines of UCLA's animal care and use committee and the National Institutes of Health Guide for the Care and Use of Laboratory Animals. Studies performed are in accordance with humane treatment of the animals.

### Echocardiography

Transthoracic echocardiography was performed at baseline and 60 days post injury using the Vevo 770 high resolution ECHO system equipped with a 35 MHz transducer. Chest fur from mice were removed and then animals were anesthetized with vaporized isoflurane (2.5% for induction, 1.0% for maintenance) in oxygen and body temperature maintained at 37° C. using a heating pad. Heart rates were maintained between 500-600 beats per minute throughout the imaging period. The probe was placed along the short axis of the left ventricle with the papillary muscles providing a guide for the proper depth. 2D images were captured to measure internal wall dimensions during both systole and diastole. Images were analyzed using the Vevo 2100 software. The left ventricle (LV) chamber dimensions and posterior wall thickness were obtained from M-mode images; LV systolic function was also assessed from these measurements by calculating ejection fraction (EF, stroke volume/end diastolic volume) and fractional shortening (FS).

### Heart Weight, Body Weight, and Tibia Length Measurements

Sixty days post-IR injury, mice from each experimental group were sacrificed and their body weights recorded. Hearts were removed, perfused in PBS, and wet weights measured. Additionally, the right tibia of each mouse was removed and measured with a caliper.

### Tissue Processing

Hearts were harvested, perfused and incubated in 4% (vol/vol) paraformaldehyde (12-18 hours at 4° C.) followed by incubation in 30% (wt/vol) sucrose in PBS at 4° C. for 12-18 hours. The samples were removed from the sucrose solution and tissue blocks were prepared by embedding in Tissue Tek O.C.T. (Sakura Finetek, 4583). Blocks were kept frozen in −80° C. Frozen whole heart blocks were sectioned into 7-10 μm thick sections with a Leica CM1860 cryostat and mounted on Superfrost/Plus slides (Fisher Scientific, 12-550-016).

### Histology and Immunofluorescence

Masson's trichrome staining (Sigma, HT15-1KT) was performed according to the manufacturer's instructions and images were taken of the entire cross-section of the heart using bright-field microscopy (Leica). ImageJ software³⁵ was used to quantify fibrosis area by comparing the area of blue (collagen) staining to the total pink/red (normal tissue) area of the left ventricle. For immunohistochemistry, sections were washed three times with PBS followed by permeabilization with 0.25% TritonX (Fisher Scientific, BP151-100) for 10 minutes. Samples were blocked for 30 min in 10% goat serum/PBS followed by incubation with primary antibodies overnight at 4° C. Antibodies against α-sarcomeric Actinin (1:400, Sigma, A7811), pHH3 (1:800, Cell Signaling Technology, 9701S), and periostin (1:150, R&D Systems, AF2955) were used. Secondary antibodies (1:150, Invitrogen) were incubated for 1 hour at RT. Isolectin B4 (Vector Laboratories, DL-1207) was used to visualize vascular density. Slides were mounted with DAPI-containing mounting media (Vector, H-1200).

### In Vitro Hypoxia Assay

HL-1 cardiomyocytes (Sigma, cat. SCC065) were maintained in growth media consisting of 10% FBS in Claycomb Media (Sigma, cat. 51800C) supplemented with 10 mM norepinephrine (Sigma, cat. A0937) and 200 mM L-glutamine (Gibco, cat. 25030081). Primary cardiac fibroblasts were isolated from Col1a1GFP/+ mice 2 months of age. Mice were injected with heparin prior to sacrifice and the heart collected. The tissue was then cut into 1-2 mm pieces and digested with Liberase Blendzyme TH and TM (Roche, cat. LIBTM-RO) in Hank's Balanced Salt Solution (HBSS) (Gibco, cat. 1417507) supplemented with DNAse I and polaxamer (10 mg/ml, Sigma, cat. 16758) in 37° C. for 1 h. Cells were passed through a 100 μm cell strainer and centrifuged at 300 g for 5 min. Cells were resuspended in 20% FBS/DMEM and transferred to a 10 cm dish precoated with 0.1% gelatin. Media was changed the next day to 10% FBS/DMEM and cells maintained in this media until start of experiment. HL-1 or Col1a1 fibroblasts were seeded into 96-well plates at a density of 17,000 cells/well and 5,000 cells/well, respectively. 24 hours after plating, cells were switched to serum starvation (1% serum) media and placed in a hypoxia chamber flushed with 1% O₂/10% CO2/balanced N2 for 3 min at a rate of 4 L/min. After 6 hours, cells were removed from the chamber and media switched to either normal growth media or media containing recombinant protein with or without EdU.

### EdU and Live/Dead Assay

EdU incorporation and detection were performed according to manufacturer's instructions (Invitrogen, C10640). Cells were labeled with EdU diluted in PBS (5 μM). After labeling period, cells were fixed in 4% paraformaldehyde followed by permeabilization with 0.5% Triton X-100. Wells were washed twice with 3% BSA in PBS. The Click-iT reaction cocktail was prepared according to instructions and cells were incubated with this solution for 30 min at room temperature. Cells were washed with 3% BSA in PBS to remove the reaction cocktail and DAPI added for imaging. To label live and dead cells, calcein AM (Thermo Fisher Scientific, C3099) and DAPI, respectively, were added to growth media and incubated for 10 minutes prior to imaging.

### Imaging Acquisition and Quantification

Fluorescent images were acquired with Leica fluorescence inverted microscope DMI6000B equipped with an EL6000 light source. For 96-well hypoxia assay, 5 images per well were taken at 10× magnification, with 4 technical replicates per treatment condition. Image quantification was performed using Imager s “threshold” and “analyze particles” functions. Assessment of live/dead was determined as the number of DAPI+ cells/HPF. EdU and pHH3 quantification were recorded as the number of EdU+ or pHH3+ nuclei over the total number of DAPI.

### BrdU and Annexin Flow Cytometry

I/R injury was performed as described above on C57BL/6 mice six months of age and 200 μl of plasma or saline were administered intravenously on day of surgery and for 4 consecutive days following. Mice were supplied BrdU in their drinking water (1 mg/mL) for 3 days. Intracellular staining for BrdU was performed in accordance to manufacturer's instructions (BD, 552598). In short, hearts were isolated and digested into single cells. Cells were fixed and permeabilized in Cytofix/Cytoperm Buffer (BD, 554714), followed by incubation in Cytoperm Permeabilization Buffer plus (BD, 561651). Cells were then exposed to fluorescent anti-BrdU antibody, washed, resuspended in staining buffer, and analyzed using a BD FACSAria II flow cytometer. For annexin V labeling, cells were stained with FITC-Annexin V antibody (BD, 556419). Thy1 (eBioscience, 47-0900-82) and CD31 (eBioscience, 25-0311-81) antibodies were used to gate fibroblast and endothelial cell populations, respectively.

### Single Cell RNA Sequencing

I/R injury was performed as described above on C57BL/6 mice six months of age and 200 ul of plasma or saline were administered intravenously on day of surgery and for 4 consecutive days following. Sham surgery with no injections served as control. On day 7 after surgery, hearts were collected and perfused with 30 ml each of HBSS. The left ventricle of each heart was obtained and the tissue chopped into 1-2 mm pieces and digested with Liberase Blendzyme TH and TM (Roche, cat. LIBTM-RO) in Hank's Balanced Salt Solution (HBSS) (Gibco, cat. 1417507) supplemented with DNAse I and polaxamer (10 mg/ml, Sigma, cat. 16758) in 37° C. for 45 minutes. Cells were passed through a 100 μm cell strainer, enzymes deactivated with 1 ml FBS, and centrifuged at 300 g for 3 min. Cells were resuspended in 0.04% BSA/PBS at approximately 1 million cells/ml for capture using the 10× Genomics Chromium Single Cell v3.0 platform. cDNA libraries were sequenced together on one lane of the Illumina NovaSeq.

Digital expression matrix was generated by de-multiplexing, barcode processing, and gene unique molecular index counting using the Cell Ranger v3.0 pipeline and the mouse mm10 reference genome. Cells that express less than 200 genes, and genes detected in less than 3 cells were filtered out. The Seurat 3.0 R toolkit for single cell genomics was used to analyze sequencing results. Downstream analysis was restricted to cells associated with at least 1150 unique molecular identifiers (UMIs) and less than 50 percent mitochondrial genes. Known marker genes for each cell type were used to identify cell clusters: cardiomyocytes (Tnnt2, Actn2, Myl2, Myl7), fibroblasts (Ddr2, Col1a1, Pdgfra, Tcf21), endothelial cells (Pecam1, Tek, Cdh5, Mcam), immune cells (Cd48, Cd68, Itgam, Ptprc), smooth muscle (Acta2, Cnn1, Myh11, Tagln), and pericytes (Notch3, Cspg4, Pdgfrb, Rgs5). Smooth muscle and pericytes were later grouped into one cluster labeled as MCs. ‘FindMarkers’ function was used to determine differential gene expression between clusters of interest. Pseudotime analysis was performed using Monocle 3.0.

### Mass Spectrometry

High abundance proteins were depleted from plasma samples according to manufacture instructions (ProteoExtract Albumin Removal Kit, Calbiochem, 122640). Samples were digested with trypsin (Promega, V1061) and labeled with TMT Label Reagents (TMT10plex Isobaric Label Reagent Set, ThermoFisher, 90110). Digested samples were fractionated using spin columns and elution solutions of varying percentages of acetonitrile in a 0.1% TEA solution. Nano LC was performed using the Easy-nLC1000 (Thermo Fisher Scientific, LC120) instrument followed by mass spectrometry using the Obitrap Q Exactive™ mass spectrometer (Thermo Fisher Scientific, IQLAAEGAAPFALGMBCA) with resolution set at 70000 at 400 m/z and precursor m/z range of 300-1650. MS files were analyzed and searched against mouse protein database based on the species of the samples using Maxquant (1.5.6.5). The parameters were set as follows: the protein modifications were carbamidomethylation (C) (fixed), oxidation (M) (variable); the enzyme specificity was set to trypsin; the maximum missed cleavages were set to 2; the precursor ion mass tolerance was set to 10 ppm, and MS/MS tolerance was 0.6 Da. Only high confident identified peptides were chosen for downstream protein identification analysis. Differentially abundant proteins between neonatal and aged mouse plasma were identified by comparing the average of normalized intensities within each sample group and calculating the ratio of neonatal over aged mouse plasma. Increased and decreased abundance proteins in neonatal compared to aged plasma were identified as ratios >2.0-fold and <0.5-fold, respectively. Pathway and molecular function analysis were performed using Metascape.

### Statistical Analysis

Statistical significance was determined by using student's t test (unpaired, two-tailed) or one-way ANOVA in GraphPad Prism 8 software. Results were significant at p<0.05 (*), p<0.01 (**), p<0.001 (***), and p<0.0001 (****). All error bars are depicted as SEM.

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

1. A method of treating or preventing an age-related disease in a subject, comprising administering a thymosin protein to the subject.

2. The method of claim 1, wherein the thymosin protein is a recombinant thymosin protein.

3. The method of claim 1, wherein the thymosin protein is selected from Tmsb4x (Thymosin beta 4), Tmsb10 (Thymosin beta 10), and Ptma (Prothymosin alpha).

4. The method of claim 1, wherein the thymosin protein is administered by intravenous delivery.

5. The method of claim 1, further comprising conjointly administering an additional thymosin protein to the subject.

6. The method of claim 5, wherein the additional thymosin protein is a recombinant thymosin protein.

7. The method of claim 5, wherein the additional thymosin protein is selected from Tmsb4x (Thymosin beta 4), Tmsb10 (Thymosin beta 10), and Ptma (Prothymosin alpha).

8. The method of claim 5, wherein the additional thymosin protein is administered by intravenous delivery.

9. The method of claim 1, wherein the age-related disease is heart disease.

10. The method of claim 9, wherein administering the thymosin protein prevents heart failure, promotes cardiac wound healing, and/or enhances cardiac repair in the subject.

11. The method of claim 9, wherein the heart disease is ischemic heart disease.

12. The method of claim 11, wherein administering the thymosin protein reduces scar size, improves cardiac function, and/or increases vascular density in the heart after ischemia-reperfusion injury.

13. The method of claim 9, further comprising administering an additional cardiovascular therapeutic to the subject.

14. The method of claim 1, wherein administering the thymosin protein to the subject reduces a humoral immune response.

15. The method of claim 1, wherein administering the thymosin protein to the subject inhibits cell death of cardiac cells.

16. A method of inhibiting cell death in a subject, comprising:
(a) determining whether serum of a subject comprises a level of a pro-aging factor above a threshold level; and
(b) if the level of the pro-aging factor is above the threshold level, administering a thymosin protein to the subject.

17. The method of claim 16, wherein the cells are cardiac cells.

18. The method of claim 16, wherein determining whether serum of a subject comprises a level of a pro-aging factor above a threshold level comprises measuring the level of the pro-aging factor in serum of the subject.

19. The method of claim 16, wherein the pro-aging factor is encoded by a gene selected from Crct1, Sprr1a, Serpinb1a, and Lgals3.

20. The method of claim 16, wherein the thymosin protein is a recombinant thymosin protein.

21-26. (canceled)
